(19) United States
(12) Patent Application Publication
(10) Pub. No.: US 2009/0270309 A1
(43) Pub. Date: Oct. 29, 2009

(54) USE OF LACTOFERRIN FRAGMENTS AND HYDROLYSATES

(76) Inventors: Jillian Cornish, Auckland (NZ); Ian Reginald Reid, Auckland (NZ); Kate Patricia Palmano, Palmerston North (NZ)

Correspondence Address: FISH & RICHARDSON PC, P.O. BOX 1022, MINNEAPOLIS, MN, 55440-1022, US

(21) Appl. No.: 12/089,788

(22) PCT Filed: Oct. 16, 2006
(86) PCT No.: PCT/NZ2006/000263

§ 371 Date: Jan. 9, 2009

(30) Foreign Application Priority Data
Oct. 14, 2005 (NZ) 543031

(51) Int. Cl. A61K 38/16; A61K 38/10; A61K 38/08

(52) U.S. Cl. 514/6; 514/12; 514/14; 514/13; 514/15; 514/17

(57) ABSTRACT

The present invention relates to use of at least one lactoferrin fragment or a lactoferrin hydrolysate or a mixture thereof for stimulating skeletal growth, inhibiting bone resorption, stimulating chondrocyte proliferation, stimulating osteoblast proliferation, inhibiting osteoclast development or treating or preventing a skeletal, joint or cartilage disorder.

### TECHNICAL FIELD

The-present invention relates to use of at least one lactoferrin fragment or a lactoferrin hydrolysate or a mixture thereof for stimulating skeletal growth, inhibiting bone resorption, stimulating chondrocyte proliferation, stimulating osteoblast proliferation, inhibiting osteoclast development or treating or preventing a skeletal, joint or cartilage disorder.

### BACKGROUND

Lactoferrin is an 80 kD iron-binding glycoprotein present in most exocrine fluids, including tears, bile, bronchial mucus, gastrointestinal fluids, cervico-vaginal mucus, seminal fluid, and milk. It is a major constituent of the secondary specific granules of circulating poly-morphonuclear neutrophils. The richest source of lactoferrin is mammalian milk and colostrum.

Lactoferrin circulates at a concentration of 2-7 μg/ml. It has multiple postulated biological roles, including regulation of iron metabolism, immune function, and embryonic development. Lactoferrin has anti-microbial activity against a range of pathogens including Gram positive and Gram negative bacteria, yeasts, and fungi. The anti-microbial effect of lactoferrin is based in part on its capability of binding iron, which is essential for the growth of the pathogens. Lactoferrin also inhibits the replication of several viruses and increases the susceptibility of some bacteria to antibiotics and lysozyme by binding to lipid A component of lipopolysaccharides on bacterial membranes.

Published International Application WO 03/082921 reports that a pure lactoferrin polypeptide containing no more than two metal ions per molecule is able to stimulate skeletal growth and inhibit bone resorption.

It would be desirable to provide an improved method for maintaining or improving bone health or to at least provide the public with a useful choice.

### SUMMARY OF THE INVENTION

Accordingly, in one aspect the present invention relates to use of at least one lactoferrin fragment or a lactoferrin hydrolysate or a mixture thereof in the manufacture of a composition for treating or preventing a skeletal, joint or cartilage disorder.

In one embodiment the present invention relates to use of at least one lactoferrin fragment or a lactoferrin hydrolysate or a mixture thereof in the manufacture of a composition for treating or preventing a skeletal, joint or cartilage disorder by stimulating skeletal growth, by inhibiting bone resorption, by stimulating chondrocyte proliferation, by stimulating osteoblast proliferation, by inhibiting osteoclast development, or a combination thereof.

In another aspect the present invention relates to a method of treating or preventing a skeletal, joint or cartilage disorder comprising administering to a subject in need thereof an effective amount of at least one lactoferrin fragment or a lactoferrin hydrolysate or a mixture thereof.

In one embodiment the present invention relates to a method of treating or preventing a skeletal, joint or cartilage disorder by stimulating skeletal growth, by inhibiting bone resorption, by stimulating chondrocyte proliferation, by stimulating osteoblast proliferation, by inhibiting osteoclast development, or a combination thereof.

The following embodiments may relate to any of the above aspects.

In one embodiment the present invention relates to use of a milk fraction comprising at least one lactoferrin fragment or a lactoferrin hydrolysate or a mixture thereof.

In one embodiment the skeletal disorder is osteoporosis, rheumatoid arthritis, osteoarthritis, hepatic osteodystrophy, osteomalacia, rickets, osteitis fibrosa cystica, renal osteodystrophy, osteosclerosis, osteopenia, fibrogenesis-imperfecta ossium, secondary hyperparathyrodism, hypoparathyroidism, hyperparathyroidism, chronic renal disease, sarcoidosis, glucocorticoid-induced osteoporosis, idiopathic hypercalcemia, Paget's disease, or osteogenesis imperfecta. In another embodiment the disorder is osteoporosis. In another embodiment the disorder is rheumatoid arthritis. In another embodiment the disorder is osteoarthritis. In one embodiment the joint or cartilage disorder is rheumatoid arthritis or osteoarthritis.

In one embodiment the lactoferrin fragment is a fragment selected from, or the lactoferrin hydrolysate comprises at least one fragment selected from:
(a) a truncated lactoferrin polypeptide, or
(b) an N-lobe fragment of lactoferrin, or
(c) a C-lobe fragment of lactoferrin, or
(d) a lactoferricin, or
(e) a lactoferrampin, or
(f) a fragment selected from SEQ ID NO. 5, 6, 7, 8, 9, 10, 11, 12, 13, 14, 15, 16, 17, 18, 19, 20, 21, 22, 23, 24, 25, 26, 27, 28, 29, 30, 31, 32 or 33, or
(g) a functional variant of any of (a) to (f), or
(h) a functional fragment of any of (a) to (g), or
(i) a mixture of any two or more of (a) to (h).

In one embodiment the lactoferrin fragment is a fragment selected from, or the lactoferrin hydrolysate comprises at least one fragment selected from
(a) an N-lobe fragment of lactoferrin, or
(b) a lactoferricin, or
(c) a functional variant of (a) or (b),
(d) a functional fragment of (a) or (b) or (c), or
(e) a mixture of any two or more fragments selected from (a) to (d).

In one embodiment the lactoferrin fragment is a fragment selected from, or the lactoferrin hydrolysate comprises at least one fragment selected from
(a) a C-lobe fragment of lactoferrin, or
(b) a lactoferrampin, or
(c) a functional variant of (a) or (b),
(d) a functional fragment of (a) or (b) or (c), or
(e) a mixture of any two or more fragments selected from (a) to (d).

In one embodiment the lactoferrin fragment is a fragment selected from, or the lactoferrin hydrolysate comprises at least one fragment selected from
(a) a polypeptide of SEQ ID NO. 5, 6, 7, 8, 9, 10, 11, 12, 13, 14, 15, 16, 17, 18, 19, 20, 21, 22, 23, 24, 25, 26, 27, 28, 29, 30, 31, 32 or 33, or
(b) a functional variant of SEQ ID NO. 5, 6, 7, 8, 9, 10, 11, 12, 13, 14, 15, 16, 17, 18, 19, 20, 21, 22, 23, 24,25, 26, 27, 28,29, 30, 31, 32 or 33, or
(c) a functional fragment SEQ ID NO. 5, 6, 7, 8, 9, 10, 11, 12, 13, 14, 15, 16, 17, 18, 19, 20, 21, 22, 23, 24, 25, 26, 27, 28, 29, 30, 31, 32 or 33, or
(d) a mixture of any two or more fragments selected from (a) to (c).

In one embodiment the truncated lactoferrin polypeptide is a polypeptide selected from SEQ ID NO. 1, 2, 3 and 4, truncated by at least about 10 amino acids at the N-terminus, the C-terminus or at both the N-terminus and C-terminus of the polypeptide.

In one embodiment the truncated lactoferrin polypeptide is a polypeptide of SEQ ID NO. 20, 24 or 26, or a mixture thereof.

In one embodiment the N-lobe fragment or flnctional fragment thereof is a polypeptide selected from SEQ ID NO. 5, 6, 9, 10, 12, 25, 27 and 29, or a mixture of any two or more thereof.

In one embodiment the C-lobe fragment or functional fragment thereof is a polypeptide selected from SEQ ID NO. 7, 8, 11, 18, 19, 21 and 23, or a mixture of any two of more thereof.

In one embodiment the lactoferricin fragment or functional fragment thereof is a polypeptide selected from SEQ ID NO. 13, 14, 15, 16, 17 and 28, or a mixture of any two or more thereof.

In one embodiment the lactoferrampin fragment is a polypeptide selected from SEQ ID NO. 30, 31, 32 and 33, or a mixture of any two or more thereof.

In one embodiment the hydrolysate is a full or partial enzyme hydrolysate (including but not limited to a protease, trypsin, chymotrypsin, chymosin, plasmin, pepsin, papain, peptidase, or aminopeptidase hydrolysates), a full or partial microorganism hydrolysate (including but not limited to hydrolysis by a bacterium from the genera Bacillus, Bifidus, Enterococcus, Lactobacillus, Lactococcus, Leuconostoc, Pediococcus, Propionbacter, Pseudomonas or Streptococcus or a mixture thereof), a full or partial acid hydrolysate (including but not limited to trifluoro acetate and hydrochloric acid hydrolysates), a cyanogen bromide hydrolysate, or a mixture thereof.

In one embodiment the hydrolysate is a hydrolysate of a lactoferrin polypeptide selected from the polypeptides of SEQ ID NO. 1, 2, 3 and 4, or a mixture thereof. In another embodiment the hydrolysate is a hydrolysate of at least one polypeptide selected from the polypeptides of SEQ ID NO. 5 to 33, or a mixture thereof.

In one embodiment the enzyme is selected from a protease, trypsin, chymotrypsin, chymosin, plasmin, pepsin, papain, a peptidase, an aminopeptidase or a mixture thereof. In another embodiment the enzyme is trypsin.

In one embodiment the enzyme is trypsin and the lactoferrin is a polypeptide having the amino acid sequence of SEQ ID NO. 2.

In one embodiment the hydrolysate comprises the peptides ADAVTLDGGMVF, ADAVTLDGGMVFEAGR, ADRDQYELL, ANEGLTWN, ANEGLTWNSLK, APVDAFK, CLQDGAGDVAFVK, DGKEDLIWK, DLLFKDSALGFLR, DSALGF, DSALGFLR, EKYYGYTGAFR, EPLQGAVAK, EPYFGYSGAFK, ESPQTHYY, ESPQTHYYAVAVVK, ETTVFENLPEK, FENLPEK, FGYSGAFK, FKDSALGFLR, GEADALNLDGGY, GEADALNLDGGYIY, GILRPYLSWTESLEPLQGAVAK, GSNFQLDQLQGR, GTEYVTAIANLKK, GYSGAFK, IIPMGILRPYLSWTESLEPLQGAVAK, IPSKVDSALYLGSR, KADAVTLDGGMVF, KADAVTLDGGMVF, KANEGLTWNSLK, KDSALGFLR, KGSNFQLDQLQGR, KPVTEAQSCHLAVAPNHAVVSR, LAQVPSHAVVAR, LAVAPNHAVVSR, LAVAVVK, LFGSPPGQR, LFKDSALGFLR, LGAPSITCVR, LGGRPTYEEY, LGGRPTYEEYLGTEY, LGGRPTYEEYLGTEYVTAIANLK, LGGRPTYEEYLGTEYVTAIANLKK, LGTEYVTAIANLK, LHQQALFGK, LLHQQALFGK, LRPVAAEIY, LRPVAAEIYGTK, LSWTESLEPLQGAVAK, LQGAVAK, NFQLDQLQGR, NLLFNDNTECLAK, PLQGAVAK, PQTHYYAVAVVK, PSKVDSALYLGSR, PTEGYLAVAVVK, PTYEEYLGTEYVTAIANLK, PVAAEIYGTK, PYLSWTESLEPLQGAVAK, QLDQLQGR, QVLLHQQALF, QVLLHQQALFGK, QVLLHQQALFGKNGK, SAGWIIPMGILRPY, SAGWIIPMGILRPYLSWTESLEPLQGAVAK, SFQLFGSPPGQR, SVDGIEDLIWK, SWTESLEPLQGAVAK, TESLEPLQGAVAK, TVFENLPEK, VFENLPEK, VLLHQQALFGK, VTAIANLK, WTESLEPLQGAVAK, YAVAVVK, YFGYSGAFK, YYGYTGAF and YYGYTGAFR, or a selection thereof that are able to stimulate osteoblast proliferation or inhibit osteoclast development or both. In one embodiment the hydrolysate is a tryptic hydrolysate. In one embodiment, hydrolysis is terminated by heating.

In one embodiment the hydrolysate comprises the peptides AEIYGTKESPQTHY, AENRKSSKYSSL, AKLGGRPTYE, AKLGGRPTYEE, AKNLNRED, AKNLNREDF, AQEKFGKNKSRS, ARSVDGKEDL, AVVKKANEGLTWNSL, DGGMVFEAGRDPYKLRPVA, DRDQYEL, DRTAGWNIPMGL, EAGRDPYKLRPVA, EAGRDPYKLRPVAA, EAGRDPYKLRPVAAE, EIYGTKESPQTHY, EKKADAVTL, ENLPEKADRDQ, ENLPEKADRDQY, ENLPEKADRDQYE, ENLPEKADRDQYEL, ESLEPLQG, ESLEPLQGA, ESLEPLQGAV, FEAGRDPYKLRPVA, FEAGRDPYKLRPVAA, FGKNKSRS, FGSPPGQRDL, FGSPPGQRDLL, FGSPPGQRDLLF, FKCLQDGAGDVAF, FKDSALGF, FKSETKNLL, FNDNTECL, FQLFGSPPGQRDLL, FRCLAEDVGD, GSPPGQRDLL, IAEKKADAVT, IAEKKADAVTL, IPMGI, IWKLLSKAQEKFGKNKSRS, IWKLLSKAQEKFGKNKSRSFQL, IYGTKESPQTHY, KAQEKFGKNKSRS, KDSALGF, KGEADALNL, KKADAVTL, KSETKNLL, KYYGYTGA, LECIRA, LFGSPPGQRDLL, LFKDSALGF, LKNLRE, LKNLRETAE, LNLDGGY, LPEKADRDQYE, LRIPSKVD, LRIPSKVDSA, LRIPSKVDSAL, LSKAQEKFGKNKSRS, LSKAQEKFGKNKSRSFQL, LTTLKNLRE, LTTLKNLRETAE, NLDGGY, NLDGGYI, NLNREDFRL, NLPEKADRDQ, NREDFRL, PEKADRDQ, PEKADRDQYE, PEKADRDQYEL, PPGQRDLL, PYKLRPVA, QLFGSPPGQRDLL, RSDRAAHVKQVL, RSVDGKEDL, RTAGWNIPMGL, SWTESLEPLQG, TESLEPLQG, TTLKNLRETAE, VARSVDGKEDL, VFEAGRDPYKLRPVA, VFEAGRDPYKLRPVAA, VFEAGRDPYKLRPVAAE, VKETTVF, VLKGEADAL, VSRSDRAAHVKQ, VTLDGGM, VTLDGGMV, VTLDGGMVF, VVARSVDGKEDL, VVKKANEGLTW, VVKKANEGLTWNSL, VVSRSDRAAHVKQ, VVSRSDRAAHVKQVL, WAKNLNRE, WAKNLNRED, WAKNLNREDF, WIIPMGI, WNIPMGL, YGTKESPQTHY and YLGSRY, or a selection thereof that are able to stimulate osteoblast proliferation or inhibit osteoclast development or both. In one embodiment the hydrolysate is a peptic hydrolysate. In one embodiment, hydrolysis is terminated by altering pH, preferably to about 8.0.

In one embodiment the hydrolysate comprises the peptides AEIYGTKESPQTHY, AKLGGRPTYE, AKLGGRPTYEE, AKNLNREDF, ARSVDGKEDL, AVVKKANEGLTWNSL, DGGMVFEAGRDPYKLRPVA, DRDQYEL, DRTAGWNIPMGL, EAGRDPYKLRPVA, EAGRDPYKLRPVAA, EAGRDPYKLRPVAAE, EIYGTKESPQTHY, ENLPEKADRDQ, ENLPEKADRDQY, ENLPEKADRDQYE, ENLPEKADRDQYEL, ESLEPLQG, ESLEPLQGA, ESLEPLQGAV, FEAGRDPYKLRPVA, FGSPPGQRDL, FGSPPGQRDLL, FGSPPGQRDLLF, FKCLQDGAGDVAF, FKDSALGF, FKSETKNLL, FQLFGSPPGQRDLL, FRCLAEDVGD, IAEKKADAVTL, IPMGI, IWKLLSKAQEKFGKNKSRSFQL, IYGTKESPQTHY, KDSALGF, KGEADALNL, KSETKNLL, KYYGYTGA, LFGSPPGQRDLL, LFKDSALGF, LKNLRETAE, LRIPSKVDSA, LRIPSKVDSAL, LSKAQEKFGKNKSRSFQL, LTTLKNLRE, LTTLKNLRETAE, NLDGGYI, NREDFRL, PEKADRDQ, PEKADRDQYE, PEKADRDQYEL, PPGQRDLL, PYKLRPVA, QLFGSPPGQRDLL, RSVDGKEDL, RTAGWNIPMGL, SWTESLEPLQG, TESLEPLQG, VFEAGRDPYKLRPVA, VFEAGRDPYKLRPVAA, VFEAGRDPYKLRPVAAE, VKETTVF, VLKGEADAL, VTLDGGM, VTLDGGMV, VTLDGGMVF, VVARSVDGKEDL, VVKKANEGLTW, VVKKANEGLTWNSL, WAKNLNRE, WAKNLNRED, WAKNLNREDF, WIIPMGI, WNIPMGL, YGTKESPQTHY and YLGSRY, or a selection thereof that are able to stimulate osteoblast proliferation or inhibit osteoclast development or both. In one embodiment the hydrolysate is a peptic hydrolysate. In one embodiment, hydrolysis is terminated by heating.

In one embodiment the microorganism is selected from the genera Bacillus, Bifidus, Enterococcus, Lactobacillus, Lactococcus, Leuconostoc, Pediococcus, Propionbacter, Pseudomonas, Streptococcus or a mixture thereof.

In one embodiment the acid is selected from trifluoro acetate and hydrochloric acid.

In one embodiment the lactoferrin fragment is a human lactoferrin fragment or a bovine lactoferrin fragment or mixtures thereof. In another embodiment the lactoferrin hydrolysate is a human lactoferrin hydrolysate or a bovine lactoferrin hydrolysate or mixtures thereof.

In one embodiment the lactoferrin fragment is naturally derived, recombinant, synthetic or a mixture thereof. In one embodiment the lactoferrin fragment is a recombinant human lactoferrin fragment. In one embodiment the lactoferrin hydrolysate is a hydrolysate of a natural, recombinant or synthetic lactoferrin polypeptide or a mixture thereof.

In one embodiment the lactoferrin or lactoferrin fragment is non-glycosylated or glycosylated. In one embodiment the lactoferrin is fully or partially glycosylated with naturally occurring or non-naturally occurring human or bovine glycosyl groups.

In one embodiment the milk fraction is a bovine milk fraction or a hydrolysed bovine milk fraction.

In one embodiment the composition or milk fraction comprises about 50 to 100% by weight, or at least about 50, 55, 60, 65, 70, 75, 80, 85, 90, 95 or 99% by weight, of at least one lactoferrin fragment or a lactoferrin hydrolysate or a mixture thereof. In another embodiment the composition or milk fraction comprises about 60 to 100% by weight, or at least about 60, 65, 70, 75, 80, 85, 90, 95 or 99% by weight, of at least one lactoferrin fragment or a lactoferrin hydrolysate or a mixture thereof. In another embodiment the composition or milk fraction comprises about 70 to 100% by weight, or at least about 70, 75, 80, 85, 90, 95 or 99% by weight, of at least one lactoferrin fragment or a lactoferrin hydrolysate or a mixture thereof. In another embodiment the composition or milk fraction comprises about 80 to 100% by weight, or at least about 80, 85, 90, 95 or 99% by weight, of at least one lactoferrin fragment or a lactoferrin hydrolysate or a mixture thereof.

In one embodiment the lactoferrin fragment comprises a metal ion binding site that is bound to a metal ion. In one embodiment the lactoferrin fragment comprises two metal ion binding sites that are independently empty or bound to a metal ion. In one embodiment the metal ion is selected from a bismuth ion, iron ion, copper ion, chromium ion, cobalt ion, manganese ion or zinc ion. In one embodiment the metal ion is an iron ion.

In one embodiment the composition is a local dosage form, an oral dosage form, a neutraceutical or a pharmaceutical. In one embodiment the lactoferrin fragment, lactoferrin hydrolysate, milk fraction or mixture thereof is administered locally or orally or parenterally.

In one embodiment a milk fraction, lactoferrin fragment or lactoferrin hydrolysate for use according to the invention may be in the form of a food, food additive, food supplement, medical food, drink, drink additive, nutraceutical or pharmaceutical composition. These compositions may include any edible consumer product which is able to carry protein. Examples of suitable edible consumer products include confectionary products, reconstituted fruit products, snack bars, muesli bars, spreads, dips, diary products including yoghurts and cheeses, drinks including dairy and non-dairy based drinks, milk powders, sports supplements including dairy and non-dairy based sports supplements, food additives such as protein sprinkles and dietary supplement products including daily supplement tablets. Suitable nutraceutical compositions useful herein may be provided in similar forms.

In one embodiment these compositions may further include another bone-enhancing agent, such as calcium, zinc, magnesium, vitamin C, vitamin D, vitamin E, vitamin K2, or a mixture thereof.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

### BRIEF DESCRIPTION OF THE DRAWINGS

FIG. 1 is a graph showing the osteoblast proliferative effects of the recombinant human lactoferrin (rhLf) N-lobe fragment (SEQ ID NO. 5) compared to full-length recombinant human lactoferrin (μg/ml). * denotes significantly different from control p<0.05 (by ANOVA, post hoc Dunnett's test).

FIG. 2 is a graph showing the osteoblast proliferative effects of a bovine lactoferricin (SEQ ID NO. 16, American Peptide Company, USA) [ANOVA, P<0.006; LFC_{—}0.1 ug/ml P<0.05 (post hoc Dunnett's test); LFC_{—}10 ug/ml P<0.01 (post hoc Dunnett's test)] compared to a human lactoferrin N-lobe fragment (SEQ ID NO. 12, Bachem, Switzerland) [ANOVA, P<0.019; nLF_{—}0.1 ug/ml and nLF_{—}1 ug/ml P<0.05 (post hoc Dunnett's test)].

FIG. 3 is a graph showing that a bovine C-lobe fragment (SEQ ID NO. 8) and cleaved but unseparated bovine N- and C-lobes (SEQ ID NO.s 8, 9 10 and 11) are also mitogenic to primary osteoblasts (x-axis units are M, molarity). * denotes significantly different from control p<0.05 (by ANOVA, post hoc Dunnett's test).

FIG. 4 is a graph showing a bovine N-lobe (SEQ ID NO. 6) and bovine C-lobe (SEQ ID NO. 7) are both mitogenic to primary osteoblasts (μg/ml). * denotes significantly different from control p<0.05 (by ANOVA, post hoc Dunnett's test).

FIG. 5 is a graph showing a synthetic bovine lactoferricin peptide is mitogenic to primary osteoblasts (SEQ ID NO. 17) (μg/ml). * denotes significantly different from control p<0.05 (by ANOVA, post hoc Dunnett's test).

FIG. 6 is a graph showing inhibition of osteoclast development in a bone marrow culture when exposed to full length recombinant human lactoferrin or a recombinant N-lobe fragment of full length recombinant human lactoferrin (SEQ ID NO. 5). OPG is osteoprotegerin, a positive inhibitor control. ANOVA P<0.0001; (Dunnett's) P<0.01 for all *.

FIG. 7 is a graph showing inhibition of osteoclast development in a bone marrow culture when exposed to a lactoferrin C-lobe fragment (SEQ ID NO. 8). OPG is osteoprotegerin, a positive inhibitor control. ANOVA, P<0.0001; C-lobe_{—}50 ug/ml P<0.05 (post hoc Dunnett's test); OPG_{—}10 ng/ml P<0.01 (post hoc Dunnett's test).

FIG. 8 is a graph showing that a tryptic hydrolysate of bovine lactoferrin (SEQ ID NO. 2) is also mitogenic to primary osteoblasts. * denotes significantly different from control p<0.03 (by ANOVA, post hoc Dunnett's test).

FIG. 9 is a graph showing that a synthetic bovine lactoferrampin (SEQ ID NO. 33) is also mitogenic to primary osteoblasts. * denotes significantly different from control p<0.02 (by ANOVA, post hoc Dunnett's test).

FIG. 10 is a graph showing that a synthetic bovine lactoferricin (SEQ ID NO. 14) is also mitogenic to primary osteoblasts. * denotes significantly different from control p<0.01 (by ANOVA, post hoc Dunnett's test).

FIG. 11 is a graph showing that a bovine lactoferrampin (SEQ ID NO. 33), a tryptic hydrolysate of SEQ ID NO. 2 and a peptic hydrolysate of SEQ ID NO. 2 are mitogenic to primary osteoblasts. * denotes significantly different from control.

FIG. 12 is a graph showing that a bovine lactoferricin (SEQ ID NO. 14) is mitogenic to primary osteoblasts at 0.01 μg/ml. * denotes significantly different from control.

### DETAILED DESCRIPTION

This invention is based on the unexpected discovery that several lactoferrin fragments and lactoferrin hydrolysates are useful in stimulating skeletal growth, inhibiting bone resorption, stimulating chondrocyte proliferation, stimulating osteoblast proliferation, inhibiting osteoclast development or treating or preventing a skeletal, joint or cartilage disorder, or a combination thereof.

### 1. Definitions

The term “comprising” as used in this specification and claims means “consisting at least in part of”. When interpreting statements in this specification and claims that include that term, the features, prefaced by that term in each statement, all need to be present but other features can also be present. Related terms such as “comprise” and “comprised” are to be interpreted in the same manner.

An “effective amount” is the amount required to confer therapeutic effect. The interrelationship of dosages for animals and humans (based on milligrams per meter squared of body surface) is described by Freireich, et al. (1966). Body surface area can be approximately determined from height and weight of the subject. See, e.g., Scientific Tables, Geigy Pharmaceuticals, Ardley, N.Y., 1970, 537. Effective doses also vary, as recognized by those skilled in the art, dependent on route of administration, excipient usage, and the like.

The term “functional fragment” is intended to mean a lactoferrin polypeptide fragment that has activity when assayed according the examples below and is able to stimulate skeletal growth, inhibit bone resorption, stimulate chondrocyte proliferation, stimulate osteoblast proliferation, or inhibit osteoclast development, or a combination thereof.

The term “functional hydrolysate” is intended to mean a full or partial lactoferrin polypeptide hydrolysate that has activity when assayed according the examples below and is able to stimulate skeletal growth, inhibit bone resorption, stimulate chondrocyte proliferation, stimulate osteoblast proliferation, or inhibit osteoclast development, or a combination thereof.

The term “functional variant” is intended to mean a variant of a lactoferrin fragment that has activity when assayed according the examples below and is able to stimulate skeletal growth, inhibit bone resorption, stimulate chondrocyte proliferation, stimulate osteoblast proliferation, or inhibit osteoclast development, or a combination thereof.

The term “glycosylated” when used in relation to a lactoferrin polypeptide or fragment is intended to mean that the lactoferrin is fully or partially glycosylated with naturally occurring or non-naturally occurring human or bovine glycosyl groups. Glycosylated and aglycosyl forms of lactoferrin are known (see Pierce, et al. (1991); Metz-Boutigue, et al. (1984); van Veen, et al. (2004)).

The term “lactoferrampin” refers to residues 268 to 284 of SEQ ID NO. 2 (²⁶⁸WKLLSKAQEKFGKNKSR²⁸⁴-SEQ ID NO. 30) and fragments thereof described by van der Kraan et al., (2004). Lactoferrampin fragments include but are not limited to ²⁶⁸WKLLSKAQEKF²⁷⁸ (SEQ ID NO. 31), ²⁷⁹GKNKSR²⁸⁴ (SEQ ID NO. 32) and ²⁶⁸WKLLSKAQEKFGKNKS²⁸³ (SEQ ID NO. 33) of SEQ ID NO. 2.

The term “lactoferricin” is intended to mean an N-terminal lactoferrin fragment. “Bovine lactoferricin” generally refers to residues 17 to 41 or 17 to 42 of bovine lactoferrin (SEQ ID NO. 2), that is FKCRRWQWRMKKLGAPSITCVRRAF (SEQ ID NO. 13) or FKCRRWQWRMKKLGAPSITCVRRAFA (SEQ ID NO. 14) (Hwang, et al. (1998); Kuwata et al., (1998)). “Human lactoferricin” generally refers to residues 1-47 of human lactoferrin (SEQ ID NO. 4), that is GRRRRSVQWCAVSQPEATKCFQWQRNMRKVRGPPVSCIKRDSPIQCI (SEQ ID NO. 15) (Bellamy, et al. (1992)).

The term “lactoferrin fragment” is intended to mean a non-glycosylated or glycosylated polypeptide sequence which comprises a naturally occurring or non-naturally occurring portion of a lactoferrin polypeptide and includes truncated wild type lactoferrin polypeptides. Useful lactoferrin fragments include individual components of hydrolysates of lactoferrin, fragments that include either or both the N and C lobe (the N- and C-terminal metal ion-binding portions of lactoferrin, respectively; Baker, et al. (2002)), fragments of the N- or C-lobes, lactoferricin (Hwang, et al. (1998); Kuwata, et al. (1998); Bellamy, et al. (1992)) and fragments generated (by artificial or natural processes) and identified by known techniques as discussed below. Useful fragments are described in greater detail below.

The term “lactoferrin hydrolysate” is intended to mean any full or partial enzyme hydrolysate (including but not limited to a protease, trypsin, chymotrypsin, chymosin, plasmin, pepsin, papain, peptidase, and aminopeptidase hydrolysates) or acid hydrolysate or a mixture thereof of a full length lactotransferrin or lactoferrin molecule or the N or C lobes thereof or mixtures thereof. Useful hydrolysates are described in greater detail below.

In one embodiment the hydrolysate is a full or partial enzyme hydrolysate (including but not limited to a protease, trypsin, chymotrypsin, chymosin, plasmin, pepsin, papain, peptidase, or aminopeptidase hydrolysates), a full or partial microorganism hydrolysate (including but not limited to hydrolysis by a bacterium from the genera Bacillus, Bifidus, Enterococcus, Lactobacillus, Lactococcus, Leuconostoc, Pediococcus, Propionbacter, Pseudomonas or Streptococcus or a mixture thereof), a full or partial acid hydrolysate (including but not limited to trifluoro acetate and hydrochloric acid hydrolysates), a cyanogen bromide hydrolysate, or a mixture thereof. In one embodiment the hydrolysate consists essentially of or consists of partially or fully hydrolysed lactoferrin.

The term “lactoferrin polypeptide” refers to non-glycosylated or glycosylated amino acid sequence of SEQ ID NO. 1, SEQ ID NO. 2, SEQ ID NO. 3 or SEQ ID NO. 4 or corresponding sequences from other species such as those described below.

The term “treat” and its derivatives should be interpreted in their broadest possible context. The term should not be taken to imply that a subject is treated until total recovery.

The term “variant” refers to a naturally occurring (an allelic variant, for example) or non-naturally occurring (an artificially generated mutant, for example) lactoferrin polypeptide or lactoferrin fragment that varies from the predominant wild-type amino acid sequence of a lactoferrin polypeptide of a given species (such as those listed below) or fragment thereof by the addition, deletion or substitution of one or more amino acids. Methods for generating such variants are known in the art and discussed below. Useful recombinant lactoferrins and lactoferrin fragments and methods of producing them are reported in U.S. patent specifications U.S. Pat. No. 5,571,691, U.S. Pat. No. 5,571,697, U.S. Pat. No. 5,571,896, U.S. Pat. No. 5,766,939, U.S. Pat. No. 5,849,881, U.S. Pat. No. 5,849,885, U.S. Pat. No. 5,861,491, U.S. Pat. No. 5,919,913, U.S. Pat. No. 5,955,316, U.S. Pat. No. 6,066,469, U.S. Pat. No. 6,080,599, U.S. Pat. No. 6,100,054, U.S. Pat. No. 6,111,081, U.S. Pat. No. 6,228,614, U.S. Pat. No. 6,277,817, U.S. Pat. No. 6,333,311, U.S. Pat. No. 6,455,687, U.S. Pat. No. 6,569,831, U.S. Pat. No. 6,635,447, US 2005-0064546 and US 2005-0114911. Useful variants also include bovine lactoferrin variants bLf-a and bLf-b (Tsuji, et al. (1989); Yoshida, et al. (1991)). Further useful variants include glycosylated and aglycosyl forms of lactoferrin (Pierce, et al. (1991); Metz-Boutigue, et al. (1984); van Veen, et al. (2004)) and glycosylation mutants.

Generally, polypeptide sequence variant possesses qualitative biological activity in common when assayed according to the examples below. Further, these polypeptide sequence variants may share at least 50%, 55%, 60%, 65%, 70%, 75%, 80%, 85%, 90%, 95%, 96%, 97%, 98% or 99% sequence identity. Also included within the meaning of the term “variant” are homologues of lactoferrin polypeptides. A homologue is typically a polypeptide from a different species but sharing substantially the same biological function or activity as the corresponding polypeptide disclosed herein.

Variant lactoferrin fragments for use according to the present invention may be generated by techniques including but not limited to techniques for mutating wild type proteins (see Sambrook, et al. (1989) and elsewhere of a discussion of such techniques) such as but not limited to site-directed mutagenesis of wild type lactoferrin and expression of the resulting polynucleotides; techniques for generating expressible polynucleotide fragments such as PCR using a pool of random or selected primers; techniques for full or partial proteolysis or hydrolysis of wild type or variant lactoferrin polypeptides; and techniques for chemical synthesis of polypeptides. Variants or fragments of lactoferrin may be prepared by expression as recombinant molecules from lactoferrin DNA or RNA, or variants or fragments thereof. Nucleic acid sequences encoding variants or fragments of lactoferrin may be inserted into a suitable vector for expression in a cell, including eukaryotic cells such as but not limited to Aspergillus or bacterial cells such as but not limited to E. coli. Lactoferrin variants or fragments may be prepared using known PCR techniques including but not limited to error-prone PCR and DNA shuffling. Error-prone PCR is a process for performing PCR under conditions where the copying fidelity of the DNA polymerase is low, such that a high rate of point mutations is obtained along the entire length of the PCR product (Leung, et al. (1989); Cadwell, et al. (1992)). DNA shuffling refers to forced homologous recombination between DNA molecules of different but highly related DNA sequence in vitro, caused by random fragmentation of the DNA molecule based on sequence homology, followed by fixation of the crossover by primer extension in a PCR reaction (Stemmer (1994)). Variants or fragments of lactoferrin may also be generated by known organic synthetic methods.

Metal ion-binding fragments of lactoferrin may be obtained by known techniques for isolating metal-binding polypeptides including but not limited to metal affinity chromatography, for example. Fragments of lactoferrin may be contacted with free or immobilised metal ions, such as Fe³⁺ and purified in a suitable fashion. For example, fragments may be contacted at neutral pH with a metal ion immobilised by chelation to a chromatography matrix comprising iminodiacetic acid or tris(carboxymethyl)ethylenediamine ligands. Bound fragments may be eluted from the supporting matrix and collected by reducing the pH and ionic strength of the buffer employed. Metal-bound fragments may be prepared according to the methods described below.

Functional variants, fragments and hydrolysates of lactoferrin may be obtained by selecting variants, fragments and hydrolysates of lactoferrin and assessing their efficacy in methods of the present invention by employing the methodologies set out in the Examples described below.

Preferred variant polypeptides preferably have at least about 70, 75, 80, 85, 90, 95 or 99% identity, preferably at least about 90, 95 or 99% identity to SEQ ID NO.1, SEQ ID NO. 2, SEQ ID NO. 3 or SEQ ID NO. 4. Variant fragments preferably have at least about 70, 75, 80, 85, 90, 95 or 99% identity, preferably at least about 90, 95 or 99% identity to a fragment described herein, including but not limited to SEQ ID NO.s 5 to 33.

Polypeptide sequence identity can be determined in the following manner. The subject polypeptide sequence is compared to a candidate polypeptide sequence using BLASTP (from the BLAST suite of programs, version 2.2.10 [October 2004]) in b12seq, which is publicly available from NCBI (ftp://ftp.ncbi.nih.gov/blast/). The default parameters of b12seq are utilized except that filtering of low complexity regions should be turned off.

Polypeptide sequence identity may also be calculated over the entire length of the overlap between a candidate and subject polynucleotide sequences using global sequence alignment programs. EMBOSS-needle (available at http:/www.ebi.ac.uk/emboss/align/) and GAP (Huang, X. (1994) On Global Sequence Alignment. Computer Applications in the Biosciences 10, 227-235.) are also suitable global sequence alignment programs for calculating polypeptide sequence identity.

Polypeptide variants also encompass those which exhibit a similarity to one or more of the specifically identified sequences that is likely to preserve the functional equivalence of those sequences and which could not reasonably be expected to have occurred by random chance. Such sequence similarity with respect to polypeptides may be determined using the publicly available b12seq program from the BLAST suite of programs (version 2.2.10 [October 2004]) from NCBI (ftp://ftp.ncbi.nih.gov/blast/).

Conservative substitutions of one or several amino acids of a described polypeptide sequence without significantly altering its biological activity are also included in the invention. A skilled artisan will be aware of methods for making phenotypically silent amino acid substitutions (see, e.g., Bowie et al., 1990).

### 2. Lactoferrin Fragments

Useful lactoferrin fragments include individual components of hydrolysates of lactoferrin, fragments that include either or both the N and C lobe (Baker, et al. (2002)), fragments of the N- or C-lobes, lactoferricin (Hwang, et al. (1998); Kuwata, et al. (1998); Bellamy, et al. (1992)) and fragments generated (by artificial or natural processes) and identified by known techniques as discussed below. Useful fragments are also described in Table 2 below. Reference in Table 2 to SEQ ID NO. 2 or 4 is intended to refer either to the full length sequence or a particular fragment defined in the ‘Residue’ column. All fragments listed in the ‘Residue’ column are believed to be useful in carrying out the claimed invention and so a fragment described with reference to certain residues of SEQ ID NO. 2 or 4 is intended to be specifically and independently disclosed as useful within the scope of the claimed invention.

Verified sequences of bovine and human lactotransferrins (lactoferrin precursors), lactoferrins and peptides therein can be found in Swiss-Prot (http://au.expasy.org/cgi-bin/sprot-search-ful).

In one embodiment the fragment or hydrolysate is a fragment or hydrolysate of the bovine lactotransferrin precursor accession number P24627 (SEQ ID NO. 1) such as the fragment bovine Lactoferricin B.

In one embodiment the fragment or hydrolysate is a fragment or hydrolysate of the human lactotransferrin precursor accession number P02788 (SEQ ID NO. 3) such as fragments Kaliocin-1, Lactoferroxin A (residues 339 to 344 of SEQ ID NO. 3-YLGSGY), Lactoferroxin B (lactoferrin residues 544 to 548 of SEQ ID NO. 3-RYYGY), and Lactoferroxin C (lactoferrin residues 681 to 687 of SEQ ID NO. 3-KYLGPQY) (see Viejo-Diaz, et al., (2003); Tani, et al., (1990)).

Other examples of lactoferrin amino acid and mRNA sequences that have been reported and are useful in carrying out the present invention include but are not limited to the amino acid (Accession Numbers AAW71443 and NP_{—}002334) and MRNA (Accession Number NM_{—}002343) sequences of human lactoferrin; the amino acid (Accession Numbers NP_{—}851341 and CAA38572) and mRNA (Accession Numbers X54801 and NM_{—}180998) sequences of bovine lactoferrin; the amino acid (Accession Numbers JC2323, CAA55517 and AAA97958) and mRNA (Accession Number U53857) sequences of goat lactoferrin; the amino acid (Accession Number CAA09407) and mRNA (Accession Number AJ010930) sequences of horse lactoferrin; the amino acid (Accession Number NP_{—}001020033) and mRNA (Accession Number NM_{—}001024862) sequences of sheep lactoferrin; the amino acid (Accession Numbers NP_{—}999527, AAL40161 and AAP70487) and mRNA (Accession Number NM_{—}214362) sequences of pig lactoferrin; the amino acid (Accession Numbers NP_{—}032548 and A28438) and mRNA (Accession Number NM_{—}008522) sequences of mouse lactoferrin; the amino acid (Accession Number CAA06441) and mRNA (Accession Number AJ005203) sequences of water buffalo lactoferrin; and the amino acid (Accession Number CAB53387) and mRNA (Accession Number AJ131674) sequences of camel lactoferrin. These sequences may be used according to the invention in wild type or variant form. Polypeptides encoded by these sequences may be isolated from a natural source, produced as recombinant proteins or produced by organic synthesis, using known techniques.

In one embodiment the lactoferrin is sheep, goat, pig, mouse, water buffalo, camel, yak, horse, donkey, llama, bovine or human lactoferrin. Preferably the lactoferrin is bovine lactoferrin.

In another embodiment the lactoferrin is recombinant sheep, goat, pig, mouse, water buffalo, camel, yak, horse, donkey, llama, bovine or human lactoferrin. Preferably the lactoferrin is recombinant bovine lactoferrin. Recombinant lactoferrin may be produced by expression in cell free expression systems or in transgenic animals, plants, fungi or bacteria, or other useful species. Alternatively, lactoferrin may be produced using known organic synthetic methods.

In yet another embodiment the lactoferrin is isolated from milk, preferably sheep, goat, pig, mouse, water buffalo, camel, yak, horse, donkey, llama, bovine or human milk. Preferably the lactoferrin is isolated from milk by cation exchange chromatography followed by ultrafiltration and diafiltration.

Preferred lactoferrin fragments include but are not limited to:
(a) a fragment having at least about 70, 75, 80, 85, 90, 95 or 99% identity to an amino acid sequence selected from SEQ ID NO. 5 to 33, or
(b) a truncated lactoferrin polypeptide comprising a polypeptide of SEQ ID NO. 1, 2, 3 or 4 truncated by about 10 to about 300 amino acids, preferably about 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295 or 300 amino acids, and including polypeptides truncated at the N-terminus, the C-terminus or at both the N-terminus and C-terminus, or
(c) an N-lobe fragment comprising residues 1 to 333 of SEQ ID NO. 4 (SEQ ID NO. 5; human), or a fragment thereof of about 10 to about 300 amino acids in length, preferably about 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295 or 300 amino acids in length, including for example a fragment selected from SEQ ID NO. 12 and 15, or
(d) an N-lobe fragment comprising residues 1 to 280 of SEQ ID NO. 2 (SEQ ID NO. 6; bovine), or residues 1 to 281 of SEQ ID NO. 2 (SEQ ID NO. 9; bovine), or residues 1 to 284 of SEQ ID NO. 2 (SEQ ID NO. 10; bovine), or a fragment of one of these sequences of about 10 to about 275 amino acids in length, preferably about 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270 or 275 amino acids in length, including for example a fragment selected from SEQ ID NO. 13, 14, 16, 17, 25, 27 and 28, or
(e) a C-lobe fragment comprising residues 345 to 689 of SEQ ID NO. 2 (SEQ ID NO. 7; bovine), or residues 285 to 689 of SEQ ID NO. 2 (SEQ ID NO. 8; bovine), or residues 283 to 689 of SEQ ID NO. 2 (SEQ ID NO. 11; bovine), or residues 342 to 689 of SEQ ID NO. 2 (SEQ ID NO. 18; bovine), or a fragment of one of these sequences of about 10 to about 400 amino acids in length, preferably about 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395 or 400 amino acids in length, including for example a fragment selected from SEQ ID NO. 19, 21 and 23, or
(f) a lactoferricin comprising SEQ ID NO.s 13 (bovine), 14 (bovine) or 15 (human), a fragment of SEQ ID NO. 13 or 14 of about 10, 15 or 20 amino acids in length such as SEQ ID NO. 16, 17 or 28, or a fragment of SEQ ID NO. 15 of about 10 to about 45 amino acids in length, preferably about 10, 15, 20, 25, 30, 35, 40 or 45 amino acids in length, or
(g) a lactoferrampin of SEQ ID NO. 30 or a fragment thereof selected from SEQ ID NO.s 31, 32 and33, or
(h) a fragment selected from SEQ ID NO. 5, 6, 7, 8, 9, 10, 11, 12, 13, 14, 15, 16, 17, 18, 19, 20, 21, 22, 23, 24, 25, 26, 27, 28, 29, 30, 31, 32 and 33.

In one embodiment the fragment may be a functional variant or function fragment of any of (a) to (h) above. One embodiment provides mixtures of any two or more of (a) to (h) or functional variants or fragments thereof. One embodiment comprises a mixture of fragments.

In one embodiment the truncated lactoferrin polypeptide is a polypeptide of SEQ ID NO. 20 (an N-terminal truncation). In another embodiment the truncated lactoferrin polypeptide is a polypeptide of SEQ ID NO. 24 or 26 (internal peptides).

In one embodiment the N-lobe fragment or functional fragment thereof is a polypeptide selected from SEQ ID NO. 5, 6, 9, 10 (N-lobes), 12, 25, 27 and 29 (N-lobe peptides), or a mixture of any two or more thereof.

In one embodiment the C-lobe fragment or functional fragment thereof is a polypeptide selected from SEQ ID NO. 7, 8, 11 (C-lobes), 18, 19, 21 and 23 (a C-lobe fragment), or a mixture of any two of more thereof.

In one embodiment the lactoferricin fragment or functional fragment thereof is a polypeptide selected from SEQ ID NO. 13, 14, 15, (lactoferricins) 16, 17 (lactoferricin peptides) and 28 (synthetic lactoferricin), or a mixture of any two or more thereof.

In one embodiment the lactoferrampin fragment is a polypeptide selected from SEQ ID NO. 30, 31, 32 and 33, or a mixture of any two or more thereof.

3. Isolation of Lactoferrin from Milk

The following is an exemplary procedure for isolating lactoferrin from bovine milk:

Fresh skim milk (7 L, pH 6.5) is passed through a 300 ml column of S Sepharose Fast Flow equilibrated in milli Q water, at a flow rate of 5 ml/min and at 4° C. Unbound protein is washed through with 2.5 bed volumes of water and bound protein eluted stepwise with approximately 2.5 bed volumes each of 0.1 M, 0.35 M, and 1.0 M sodium chloride. Lactoferrin eluting as a discreet pink band in 1 M sodium chloride is collected as a single fraction and dialysed against milli Q water followed by freeze-drying. The freeze-dried powder is dissolved in 25 mM sodium phosphate buffer, pH 6.5 and subjected to chromatography on S Sepharose Fast Flow with a sodium chloride gradient to 1 M in the above buffer and at a flow rate of 3 ml/min. Fractions containing lactoferrin of sufficient purity as determined by gel electrophoresis and reversed phase HPLC are [symbol] ed and freeze-dried. Final purification of lactoferrin is accomplished by gel filtration on Sephacryl 300 in 80 mM dipotassium phosphate, pH 8.6, containing 0.15 M potassium chloride. Selected fractions are combined, dialyzed against milli Q water, and freeze-dried. The purity of this preparation is greater than 95% as indicated by HPLC analysis and by the spectral ratio values (280 nm/465 nm) of ˜19 or less for the iron-saturated form of lactoferrin.

### 4. Iron Saturation or Depletion of Lactoferrin

Iron saturation is achieved by addition of a 2:1 molar excess of 5 mM ferric nitrilotriacetate (Foley and Bates (1987)) to a 1% solution of the purified lactoferrin in 50 mM Tris, pH 7.8 containing 10 mM sodium bicarbonate. Excess ferric nitrilotriacetate is removed by dialysis against 100 volumes of milli Q water (twice renewed) for a total of 20 hours at 4° C. The iron-loaded (holo-) lactoferrin may then be freeze-dried.

Iron-depleted (apo-) lactoferrin is prepared by dialysis of a 1% solution of the highly purified lactoferrin sample in water against 30 volumes of 0.1 M citric acid, pH 2.3, containing 500 mg/L disodium EDTA, for 30 h at 4° C. (Masson and Heremans (1966)). Citrate and EDTA are then removed by dialysis against 30 volumes of milli Q water (once renewed) and the resulting colourless solution may be freeze-dried.

A lactoferrin polypeptide can contain an iron ion (as in a naturally occurring lactoferrin polypeptide) or a non-iron metal ion (e.g., a copper ion, a chromium ion, a cobalt ion, a bismuth ion, a manganese ion, or a zinc ion). For instance, lactoferrin isolated from bovine milk can be depleted of iron and then loaded with another type of metal ion. For example, copper loading can be achieved according to the same method for iron loading described above. For loading lactoferrin with other metal ions, the method of Ainscough, et al. (1979) can be used.

In a preparation of a composition for use according to the invention, a lactoferrin polypeptide or metal ion-binding lactoferrin fragment can be of a single species, or of different species. For instance, the polypeptides or fragments can each contain a different number of metal ions or a different species of metal ions; or the lengths of the polypeptides can vary, e.g., some are full-length polypeptides and some are fragments, and the fragments can each represent a particular portion of a full-length polypeptide. Such a preparation can be obtained from a natural source or by mixing different lactoferrin polypeptide species. For example, a mixture of lactoferrin polypeptides of different lengths can be prepared by proteinase digestion (complete or partial) of full-length lactoferrin polypeptides. The degree of digestion can be controlled according to methods well known in the art, e.g., by manipulating the amount of proteinase or the time of incubation, and described below. A full digestion produces a mixture of various fragments of full-length lactoferrin polypeptides; a partial digestion produces a mixture of full-length lactoferrin polypeptides and various fragments.

### 5. Preparation of Lactoferrin Fragments or Lactoferrin Hydrolysates

In one embodiment the hydrolysate is a full or partial enzyme hydrolysate (including but not limited to a protease, trypsin, chymotrypsin, chymosin, plasmin, pepsin, papain, peptidase, or aminopeptidase hydrolysates), a full or partial microorganism hydrolysate (including but not limited to hydrolysis by a bacterium from the genera Bacillus, Bifidus, Enterococcus, Lactobacillus, Lactococcus, Leuconostoc, Pediococcus, Propionbacter, Pseudomonas or Streptococcus or a mixture thereof), a full or partial acid hydrolysate (including but not limited to trifluoro acetate and hydrochloric acid hydrolysates), a cyanogen bromide hydrolysate, or a mixture thereof.

Hydrolysates containing target peptides can be prepared by selecting suitable enzymes with known specificity of cleavage, such as pepsin, trypsin or chymotrypsin, and controlling/limiting proteolysis by pH, temperature, time of incubation and enzyme to substrate ratio. Refinement of such isolated peptides can be made using specific endopeptidases. In one embodiment, hydrolysis is terminated by heating. In another embodiment, hydrolysis is terminated by adjusting the pH. In one embodiment the enzyme is pepsin and hydrolysis is terminated by adjusting the pH to about pH 6.0 or more. In another embodiment the enzyme is trypsin and hydrolysis is terminated by adjusting the pH to less than about pH 3.0 or more than about pH 11. In another embodiment the enzyme is trypsin and hydrolysis is terminated by incubation at about 40° C. or higher. In this embodiment, the presence of peptides derived from trypsin indicates autolysis during incubation and so hydrolysis is therefore self-limiting.

As an example, bovine lactoferricin can be produced by cleavage of bovine lactoferrin with pepsin at pH 2.0 for 45 min at 37° C. (Facon & Skura, 1996), or at pH 2.5, 37° C. for 4 h using enzyme at 3% (w/w of substrate) (Tomita et al., 1994). The peptide can then be isolated by reversed phase HPLC (Tomita et al., 1994) or hydrophobic interaction chromatography (Tomita e al., 2002). Optionally, hydrolysis is terminated by adjusting the pH to 8.0, for example with NaOH.

As another example, bovine lactoferrin (SEQ ID NO. 2), 2% w/v in 0.1 M ammonium bicarbonate, pH 8.0, was hydrolysed 20 h at 35° C. with trypsin (Sigma T1426, Sigma Chemical Co., MO, USA) at an E:S ratio of 1:40. Reaction was monitored by SDS-PAGE. The hydrolysate was heated for 10 min at 80° C. to inactivate residual enzyme and the peptides recovered by freeze-drying. Peptides were identified by LC/MS/MS on an Orbitrap ESI-TRAP (Thermo Electron Corporation) (Table 1a).

Alternatively, lactoferrin peptides can be produced by well established synthetic Fmoc chemistry as described for human kaliocin-1 (NH2-FFSASCVPGADKGQFPNLCRLCAGTGENKCA-COOH) and the lactoferricin derived peptide (NH2-TKCFQWQRNMRKVRGPPVSCIKR-COOH) in Viejo-Diaz et al., (2003); and bovine lactoferricin peptide (NH2-RRWQWRMKKLG-COOH) as described in Nguyen et al., (2005); and lactoferrampin (NH2-WKLLSKAQEKFGKNKSR-COOH) and shorter fragments as described in van der Kraan et al., (2004).

In general, SDS-PAGE may be used to estimate the degree of hydrolysis by comparison of the hydrolysate to a molecular weight standard. Size exclusion chromatography may be used to separate various species within a hydrolysate and to estimate a molecular weight distribution profile.

In a preferred hydrolytic method, bovine lactoferrin was dissolved to 20 mg/mL in 50 mM Tris pH 8.0, 5 mM CaCl2. Trypsin (Sigma T8642, TPCK treated, Type XII from bovine pancreas, 11700 U/mg protein) was added at an enzyme substrate ratio of 1:50 w/w and the mixture incubated at 25° C. for 3 h. The reaction was stopped by the addition of PMSF to 1 mM final concentration and extent of digestion monitored by SDS-PAGE. The tryptic digest (4 mL) was applied to gel filtration on Sephacryl S300 (Amersham GE) (90 cm×2.6 cm column) in 50 mM Tris, 0.15M NaCl pH 8.0. Suitable fractions containing the major fragments of bovine lactoferrin (Legrand et al., 1984) were then subjected to cation exchange chromatography on S Sepharose fast Flow (Amersham GE) (15 cm×1.6 cm column) using sodium phosphate buffer pH 6.5 and a salt gradient to 1 M NaCl. Final separation of the C lobe and N+C lobes was achieved by further gel filtration on Sephacryl S300 as above but using 10% v/v acetic acid as eluent (Mata et al., 1994). The identity of the dialysed (versus milli-Q water) and freeze-dried fragments was confirmed by SDS-PAGE and Edman N-terminal sequencing.

In another method, a tryptic digest as above was separated by RP-HPLC on a Vydac C18 column as in Superti et al., (2001) and the high mass fragments corresponding to C-lobe and N-lobe fragments recovered. Identity was confirmed by MALDI MS.

### 6. Medicinal Uses and Methods of Treatment

A lactoferrin fragment or hydrolysate or mixture thereof may be used to treat or prevent skeletal, joint or cartilage disorders. Examples of such disorders include, but are not limited to osteoporosis, rheumatoid arthritis, osteoarthritis, hepatic osteodystrophy, osteomalacia, rickets, osteitis fibrosa cystica, renal osteodystrophy, osteosclerosis, osteopenia, fibrogenesis-imperfecta ossium, secondary hyperparathyrodism, hypoparathyroidism, hyperparathyroidism, chronic renal disease, sarcoidosis, glucocorticoid-induced osteoporosis, idiopathic hypercalcemia, Paget's disease, and osteogenesis imperfecta.

A nutraceutical composition for use according to the invention can be a dietary supplement (e.g., a capsule, a mini-bag, or a tablet) or a food product (e.g., milk, juice, a soft drink, a herbal tea-bag, or confectionary). The composition can also include other nutrients, such as a protein, a carbohydrate, vitamins, minerals, or amino acids. The composition can be in a form suitable for oral use, such as a tablet, a hard or soft capsule, an aqueous or oil suspension, or a syrup; or in a form suitable for parenteral use, such as an aqueous propylene glycol solution, or a buffered aqueous solution. The amount of the active ingredient in the nutraceutical composition depends to a large extent on a subject's specific need. The amount also varies, as recognized by those skilled in the art, dependent on administration route, and possible co-usage of other bone-enhancing agents.

Also within the scope of this invention is a pharmaceutical composition that contains an effective amount of at least one lactoferrin fragment or hydrolysate or a mixture thereof as described above, and a pharmaceutically acceptable carrier. The composition may contain a combination of fragments, a combination of hydrolysates or a combination of fragments and hydrolysates. The pharmaceutical composition can be used to prevent and treat bone-related disorders described above. The pharmaceutical composition can further include an effective amount of another bone-enhancing agent. The pharmaceutically acceptable carrier includes a solvent, a dispersion medium, a coating, an antibacterial and antifungal agent, and an isotonic and absorption delaying agent.

At least one lactoferrin fragment or hydrolysate or a mixture thereof as described above can be formulated into dosage forms for different administration routes utilizing conventional methods. For example, it can be formulated in a capsule, a gel seal, or a tablet for oral administration. Capsules can contain any standard pharmaceutically acceptable materials such as gelatin or cellulose. Tablets can be formulated in accordance with conventional procedures by compressing mixtures of the at least one lactoferrin fragment or hydrolysate or a mixture thereof with a solid carrier and a lubricant. Examples of solid carriers include starch and sugar bentonite. The at least one lactoferrin fragment or hydrolysate or a mixture thereof can also be administered in a form of a hard shell tablet or a capsule containing a binder, e.g., lactose or mannitol, a conventional filler, and a tabletting agent. The pharmaceutical composition can be administered via the parenteral route. Examples of parenteral dosage forms include aqueous solutions, isotonic saline or 5% glucose of the active agent, or other well-known pharmaceutically acceptable excipient. Cyclodextrins, or other solubilising agents well-known to those familiar with the art, can be utilized as pharmaceutical excipients for delivery of the therapeutic agent.

The efficacy of a composition useful according to this invention can be evaluated both in vitro and in vivo. See, e.g., the examples below. Briefly, the composition can be tested for its ability to promote osteoblast and chondrocyte proliferation or inhibit osteoclastogenesis in vitro. For in vivo studies, the composition can be injected into an animal (e.g., a mouse) and its effects on bone tissues are then accessed. Based on the results, an appropriate dosage range and administration route can be determined.

For example, foods, food additives or food supplements comprising at least one lactoferrin fragment or hydrolysate or a mixture thereof for use according to the invention include any edible consumer product which is able to carry protein. Examples of suitable edible consumer products include confectionary products, reconstituted fruit products, snack bars, muesli bars, spreads, dips, diary products including yoghurts and cheeses, drinks including dairy and non-dairy based drinks, milk powders, sports supplements including dairy and non-dairy based sports supplements, food additives such as protein sprinkles and dietary supplement products including daily supplement tablets. Suitable nutraceutical compositions useful herein may be provided in similar forms.

A suitable pharmaceutical composition may be formulated with appropriate pharmaceutically acceptable excipients, diluents or carriers selected with regard to the intended dosage form and standard pharmaceutical formulation practice. A dosage form useful herein can be administered orally as a powder, liquid, tablet or capsule. Suitable dosage forms may contain additional agents as required, including emulsifying, antioxidant, flavouring or colouring agents. Dosage forms useful herein may be adapted for immediate, delayed, modified, sustained, pulsed or controlled release of the active components.

A preferred lactoferrin composition for use herein comprises at least one lactoferrin fragment or lactoferrin hydrolysate, or a mixture of fragments or hydrolysates or both. Preferably the lactoferrin is bovine lactoferrin. Preferably the composition further comprises a digestible protein such as casein or other protective protein. Preferably the composition comprises about 0.1 to 90 wt % lactoferrin and about 10 to 90 wt % casein or other protective protein. More preferably the composition consists essentially of about 0.5 to 10 wt % lactoferrin and about 10 to 99 wt % casein or other protective protein. Most preferably the composition consists essentially of about 1 wt % lactoferrin and about 20 wt % casein or other protective protein.

At least one lactoferrin fragment or hydrolysate or a mixture thereof may also be administered by parenteral routes including but not limited to subcutaneous, intravenous, intraperitoneal, intramuscular and intratumoural administration. Preferably at least one lactoferrin fragment or hydrolysate or a mixture thereof is administered parenterally by injection. Those skilled in the art will be able to prepare suitable formulations for parenteral administration without undue experimentation.

The at least one lactoferrin fragment or hydrolysate or a mixture thereof may be used alone or in combination with one or more other therapeutic agents (nutraceuticals, pharmaceuticals or medical foods, for example). When used in combination with another therapeutic agent the administration of the two agents may be separate, simultaneous or sequential. Simultaneous administration includes the administration of a single dosage form that comprises both agents and the administration of the two agents in separate dosage forms at substantially the same time. Sequential administration includes the administration of the two agents according to different schedules, preferably so that there is an overlap in the periods during which the two agents are provided. Suitable agents with which the compositions of the invention can be co-administered include other bone growth agents or bone disease treatments, and other suitable agents known in the art. Such agents are preferably administered parenterally, preferably by intravenous, subcutaneous, intramuscular, intraperitoneal, intramedullar, epidural, intradermal, transdermal (topical), transmucosal, intra-articular, and intrapleural, as well as oral, inhalation, and rectal administration.

Additionally, it is contemplated that a composition in accordance with the invention may be formulated with additional active ingredients which may be of benefit to a subject in particular instances. For example, therapeutic agents that target the same or different facets of the disease process may be used.

As will be appreciated, the dose of the composition administered, the period of administration, and the general administration regime may differ between subjects depending on such variables as the severity of symptoms of a subject, the type of disorder to be treated, the mode of administration chosen, and the age, sex and/or general health of a subject.

It should be appreciated that administration may include a single daily dose or administration of a number of discrete divided doses as may be appropriate.

It should be understood that a person of ordinary skill in the art will be able without undue experimentation, having regard to that skill and this disclosure, to determine an effective dosage regime (including daily dose and timing of administration) for a given condition.

Various aspects of the invention will now be illustrated in non-limiting ways by reference to the following examples.

### ExampleS

### Example 1

### Lactoferrin Fragments Promote Proliferation of Primary Rat Osteoblasts

Osteoblasts were isolated by collagenase digestion from 20-day fetal rat [symbol] viously described by Lowe, et al. (1991). Calvariae were dissected aseptically, and the frontal and parietal bones were stripped of their periosteum. Only the central portions of the bones, free from suture tissue, were collected. The calvariae were treated twice with phosphate buffered saline (PBS) containing 3 mM EDTA (pH 7.4) for 15 minutes at 37° C. in a shaking water bath. After washing once in PBS, the calvariae were treated twice with 3 ml of 1 mg/ml collagenase for 7 minutes at 37° C. After discarding the supernatants from digestions I and II, the calvariae were treated further two times with 3 ml of 2 mg/ml collagenase (30 mins, 37° C.). The supernatants of digestions III and IV were pooled, centrifuged, and the cells washed in Dulbecco's modified Eagle's medium (DME) with 10% fetal calf serum (FCS), suspended in DME/10% FCS, and placed in 75 cm³ flasks. The cells were incubated under 5% CO2 and 95% air at 37° C. Confluence was reached by 5-6 days, at which time the cells were subcultured. After trypsinization using trypsin-EDTA (0.05%/0.53 mM), the cells were rinsed in minimum essential medium (MEM) with 5% FCS and resuspended in a fresh medium, then seeded at 5×104 cells/ml in 24-well plates (0.5 ml cell suspension per well, i.e., 1.4×104 cells/cm²). The osteoblast-like character of these cells has been established by demonstration of high levels of alkaline phosphatase activity and osteocalcin production (Groot, et al. (1985)) and a sensitive adenylate cyclase response to parathyroid hormone and prostaglandins (Hermann-Erlee, et al. (1986)).

Proliferation studies (cell counts and thymidine incorporation) were performed both in actively growing and non-actively growing cell populations. To produce actively growing cells, sub-confluent populations (24 h after subculturing) were placed in fresh MEM containing 1% FCS and a lactoferrin fragment sample (see Table 1 below). To produce non-actively growing cells, sub-confluent populations were placed in serum-free medium with 0.1% bovine serum albumin plus a lactoferrin fragment sample. Cell numbers were analyzed at 6, 24, and 48 hours after the addition of lactoferrin fragment samples. The cell numbers were determined after detaching cells from the wells by exposure to trypsin/EDTA (0.05%/0.53 mM) for approximately 5 minutes at 37° C. Counting was performed in a haemocytometer chamber. [3H]-thymidine incorporation into actively growing and non-actively growing cells was assessed by pulsing the cells with [3H]-thymidine (1 μCi/well) two hours before the end of the incubation. The experiment was terminated at 6, 24, or 48 hours by washing the cells in MEM containing cold thymidine followed by the addition of 10% trichloroacetic acid. The precipitate was washed twice with ethanol:ether (3:1), and the wells desiccated at room temperature. The [symbol] redissolved in 2 M KOH at 55° C. for 30 min, neutralized with 1 M HCl, and an aliquot counted for radioactivity. For both cell counts and thymidine incorporation, each experiment at each time point was performed at least 4 different times using experimental groups consisting of at least 6 wells.

The mitogenic response of the purified lactoferrin fragment samples were found to significantly increase the rate of osteoblast cell proliferation (i.e., increase in thymidine incorporation into DNA of growing cells). The osteogenic response seen above was compared with that of insulin-like growth factor 1 (IGF-1), a well-recognized osteoblast mitogen. The magnitudes of response of the lactoferrin fragments were similar to IGF-1 in the same osteoblast cell culture system.

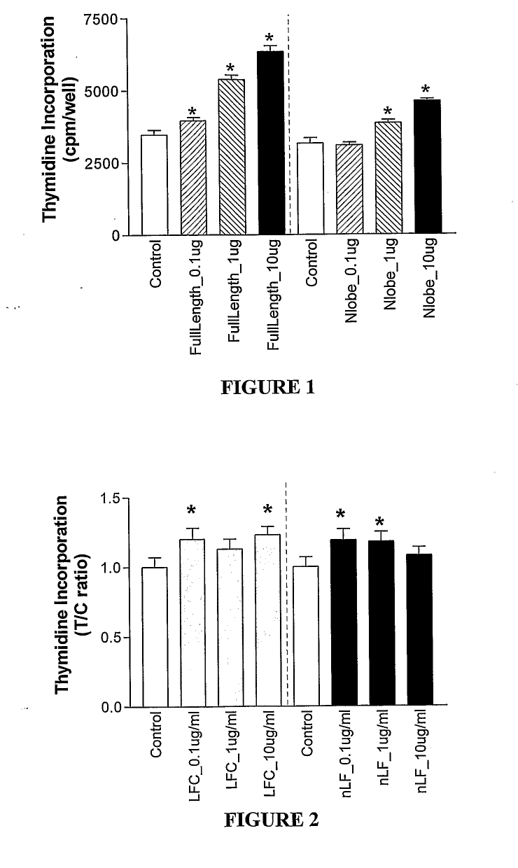

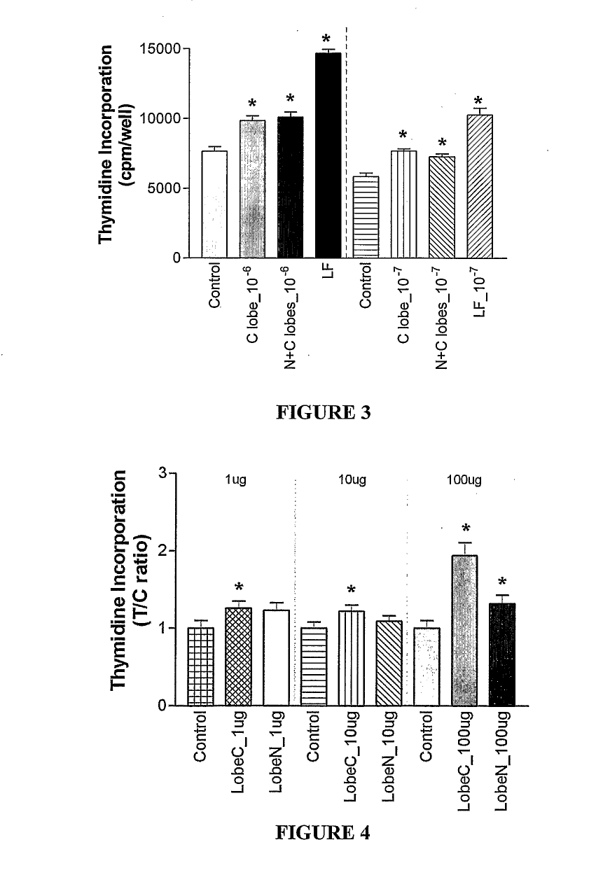

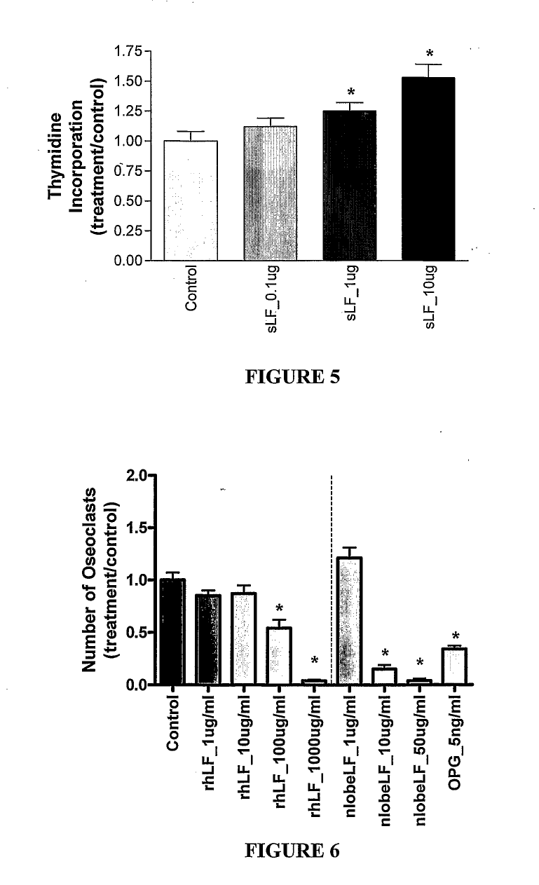

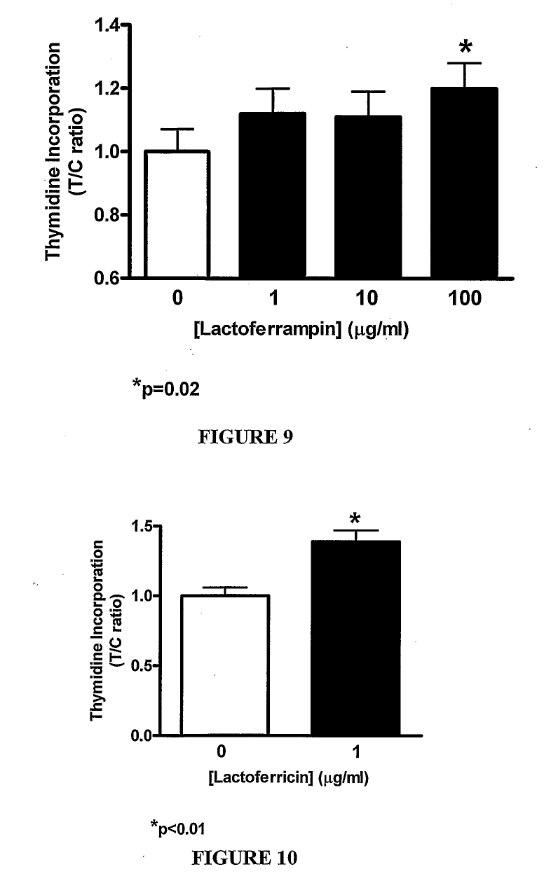

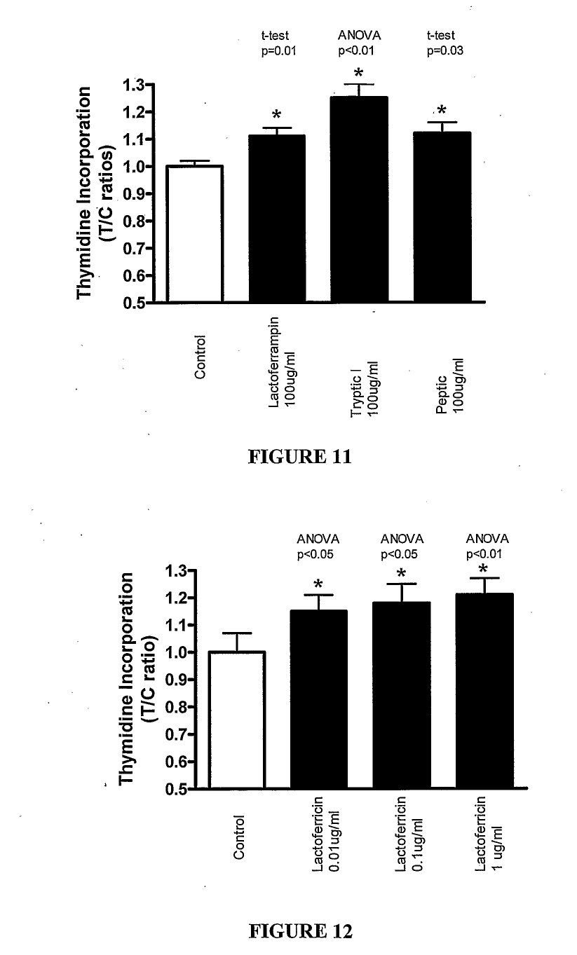

Table 1 describes the lactoferrin fragments used in Example 1 and the minimum dose range that stimulate osteoblast proliferation. Results are also shown in FIGS. 1 to 5 and 8 to 10, as described above.

TABLE 1
Lactoferrin fragments that stimulate osteoblast proliferation

| | | | | | |
|---|---|---|---|---|---|
| | | | | Dose | Dose |
| Peptide | Species | Sequence^{#} | Amino Acid Residues | (ug/mL) | (M) |

| | | | | | |
|---|---|---|---|---|---|
| N-Lobe | Recombinant | 1-333 | ¹GRRRRV . . . IQNLR³³³ | 0.1-10 | 10^{-7/8} |
| | Human (rh) | (a) | (Mᵣ~40 kDa by SDS-PAGE) | | |
| | | | SEQ ID NO. 5 | | |
| N-Lobe | Bovine | 1-280 | ¹APRKNV . . . EKFGK²⁸⁰ | 1-100 | 10^{-6/7} |
| | | (b) | (Mᵣ 31,326) | | |
| | | | SEQ ID NO. 6 | | |
| C-Lobe | Bovine | 345-689 | ³⁴⁵VVWCA . . . AFLTR⁶⁸⁹ | 1-100 | 10^{-6/7} |
| | | (b) | (Mᵣ 37,545.5) | | |
| | | | SEQ ID NO. 7 | | |
| C-Lobe | Bovine | 285-689 | ²⁸⁵SFQLFGSP . . . AFLTR⁶⁸⁹ | 1-100 | 10^{-6/7} |
| | | (c) | (Mᵣ~50 kDa by SDS-PAGE) | | |
| | | | SEQ ID NO. 8 | | |
| N + C-Lobes, | Bovine | 1- | ¹APRKNVRWCT . . . FGKNK²⁸²(SR²⁸⁴) | 10-100 | 10^{-6/7} |
| cleaved but not | | 282/284; | (Mᵣ~30 kDa by SDS-PAGE | | |
| separated | | 283/285- | SEQ ID NO. 9, SEQ ID NO. 10 | | |
| | | 689 (c) | (²⁸³SR)²⁸⁵SFQLFGSP . . . AFLTR⁶⁸⁹ | | |
| | | | (Mᵣ~50 kDa by SDS-PAGE) | | |
| | | | SEQ ID NO. 11, SEQ ID NO. 8 | | |
| N-Lobe | Synthetic | 232-246 | ²³²CPDNTRKIPVDKFKDC²⁴⁶ | 0.1, 1 | 10^{-7/8} |
| peptide | Human | (d) | (Mᵣ 1764.02) | | |
| | | | SEQ ID NO. 12 | | |
| Lactoferricin | Synthetic | 20-30 | ²⁰RRWQWRMKKLG³⁰ | 0.1-10 | 10^{-6/7/5} |
| peptide | Bovine | (e) | (Mᵣ 1544.9) | | |
| | | | SEQ ID NO. 16 | | |
| Lactoferricin | Synthetic | 17-31 | ¹⁷FKCRRWQWRMKKLGA³¹ | 0.1-10 | 10^{-6/7/8} |
| peptide | Bovine | (f) | (Mᵣ 1993) | | |
| | | | SEQ ID NO. 17 | | |
| Lactoferrin | Bovine | 1-689 | Data listed in Table 1a | 100 | 10⁻⁶ |
| tryptic | | (g) | SEQ ID NO. 2 | | |
| hydrolysate | | | | | |
| Lactoferrampin | Synthetic | 268-283 | ²⁶⁸WKLLSKAQEKFGKNKS²⁸³ | 100 | 10⁻⁵ |
| | Bovine | (h) | (Mr 1892) | | |
| | | | SEQ ID NO. 33 | | |
| Lactoferricin | Synthetic | 17-42 | ¹⁷FKCRRWQWRMKKLGAPSITCVRRAFA⁴² | 0.01-1.0 | 10^{-7/9} |
| peptide | Bovine | (h) | (Mr 3197) | | |
| | | | SEQ ID NO. 14 | | |
| Lactoferrin | Bovine | 1-689 | Data listed in Table 1b | 100 | 10⁻⁶ |
| peptic | | (i) | SEQ ID NO. 2 | | |
| hydrolysate | | | | | |

^{#}The sequences given in Table 1 are numbered according to general convention from the N-terminus excluding the signal peptide (¹MKLFVPALLSLGALGLCLA¹⁹ from SEQ ID NO. 1 or ¹MKLVFLVLLFLGALGLCLA¹⁹ from SEQ ID NO. 3) e.g. they begin at residue 20 of SEQ ID NO. 1 (¹APRKNV . . . ) or residue 20 of SEQ ID NO. 3 (¹GRRRRV . . . ). Sequences excluding the signal peptide are provided as SEQ ID NO.s 2 (bovine) and 4 (human). An example signal peptide is provided as SEQ ID NO. 22.

With reference to Table 1, letters in brackets denote:
(a) Expressed from baby hamster kidney cells (Tweedie, et al. (1994)).
(b) Tryptic cleavage and verification by MALDI-TOF mass spec (Superti, et al. (2001).
(c) Controlled tryptic hydrolysis of bovine lactoferrin to yield two major cleavage fragments as seen by SDS-PAGE. ‘C-Lobe’ isolated by size exclusion and ion exchange chromatography, identity verified by Edman sequencing to 10 aa residues, and size by SDS-PAGE. For N+C Lobe, ‘N-lobe’ identity verified by Edman sequencing to 10 amino acid residue, and size by SDS-PAGE; ‘C-Lobe’ identity verified by Edman sequencing to 10 aa residues, and by size. Presence of an extra cleavage point at K282S283, determined by Edman sequencing. See Legrand, et al. (1984).
(d) Manufactured by Bachem (Switzerland).
(e) Manufactured by American Peptide Company (USA). See also Tomita, et al. (1994) and Vogel, et al. (2002).
(f) Manufactured by Auspep (Australia). See also Tomita, et al. (1994) and Vogel, et al. (2002).
(g) Bovine lactoferrin (SEQ ID NO. 2), 2% w/v in 0.1 M ammonium bicarbonate, pH 8.0, was hydrolysed 20 h at 35° C. with trypsin (Sigma T1426, Sigma Chemical Co., MO, USA) at an E:S ratio of 1:40. Reaction was monitored by SDS-PAGE. The hydrolysate was heated for 10 min at 80° C. to inactivate residual enzyme and the peptides recovered by freeze-drying. Peptides were identified by LC/MS/MS on an Orbitrap ESI-TRAP (Thermo Electron Corporation) (Table 1a). The list of peptides identified does not necessarily exclude other peptides which might have been present but not detected under the analytical conditions, or not validated. Peptides were validated if the peptide score indicated homology or identity, and only if there were 5 consecutive ‘y’ series or ‘b’ series ions in the MS/MS data, or for short sequences, at least two sets of 4 consecutive ‘y’ series and/or ‘b’ series ions.
(h) Manufactured by Auspep (Australia).
(i) Bovine lactoferrin (SEQ ID 2) was dissolved to 1 %(w/v) in milliQ water, and the pH adjusted to 2.0 with HCl. Pepsin (Sigma P7012) was added at an E:S of 1:100 and hydrolysis was continued for 20 h at 35° C., with monitoring by SDS-PAGE. The hydrolysis was terminated by adjusting the pH to 8.0 with NaOH and peptides recovered by freeze-drying. Peptides were identified by LC/MS/MS on an Orbitrap ESI-TRAP (Thermo Electron corporation) (Table 1b). The list of peptides does not necessarily exclude other peptides which might have been present but not detected under the analytical conditions.

TABLE 1a
Peptides present in a tryptic hydrolysate
of SEQ ID NO. 2

| Peptide | Residues^{#} |
|---|---|
| ADAVTLDGGMVF | 73-84 |
| ADAVTLDGGMVFEAGR | 73-88 |
| ADRDQYELL | 241-249 |
| ANEGLTWN | 461-468 |
| ANEGLTWNSLK | 461-471 |
| APVDAFK | 256-262 |
| CLQDGAGDVAFVK | 217-229 |
| DGKEDLIWK | 280-288 |
| DLLFKDSALGFLR | 316-328 |
| DSALGF | 321-326 |
| DSALGFLR | 321-328 |
| EKYYGYTGAFR | 540-550 |
| EPLQGAVAK | 162-170 |
| EPYFGYSGAFK | 206-216 |
| ESPQTHYY | 105-112 |
| ESPQTHYYAVAVVK | 105-118 |
| ETTVFENLPEK | 230-240 |
| FENLPEK | 234-240 |
| FGYSGAFK | 209-216 |
| FKDSALGFLR | 319-328 |
| GEADALNLDGGY | 406-417 |
| GEADALNLDGGYIY | 406-419 |
| GILRPYLSWTESLEPLQGAVAK | 149-170 |
| GSNFQLDQLQGR | 120-131 |
| GTEYVTAIANLKK | 681-693 |
| GYSGAFK | 210-216 |
| IIPMGILRPYLSWTESLEPLQGAVAK | 145-170 |
| IPSKVDSALYLGSR | 329-342 |
| KADAVTLDGGMVF | 72-84 |
| KADAVTLDGGMVF | 72-84 |
| KANEGLTWNSLK | 460-471 |
| KDSALGFLR | 320-328 |
| KGSNFQLDQLQGR | 119-131 |
| KPVTEAQSCHLAVAPNHAVVSR | 598-619 |
| LAQVPSHAVVAR | 266-277 |
| LAVAPNHAVVSR | 608-619 |
| LAVAVVK | 453-459 |
| LFGSPPGQR | 307-315 |
| LFKDSALGFLR | 318-328 |
| LGAPSITCVR | 48-57 |
| LGGRPTYEEY | 670-679 |
| LGGRPTYEEYLGTEY | 670-684 |
| LGGRPTYEEYLGTEYVTAIANLK | 670-692 |
| LGGRPTYEEYLGTEYVTAIANLKK | 670-693 |
| LGTEYVTAIANLK | 680-692 |
| LHQQALFGK | 631-639 |
| LLHQQALFGK | 630-639 |
| LRPVAAEIY | 93-101 |
| LRPVAAEIYGTK | 93-104 |
| LSWTESLEPLQGAVAK | 155-170 |
| LQGAVAK | 164-170 |
| NFQLDQLQGR | 122-131 |
| NLLFNDNTECLAK | 657-669 |
| PLQGAVAK | 163-170 |
| PQTHYYAVAVVK | 107-118 |
| PSKVDSALYLGSR | 330-342 |
| PTEGYLAVAVVK | 448-459 |
| PTYEEYLGTEYVTAIANLK | 674-692 |
| PVAAEIYGTK | 95-104 |
| PYLSWTESLEPLQGAVAK | 153-170 |
| QLDQLQGR | 124-131 |
| QVLLHQQALF | 628-637 |
| QVLLHQQALFGK | 628-639 |
| QVLLHQQALFGKNGK | 628-642 |
| SAGWIIPMGILRPY | 141-154 |
| SAGWIIPMGILRPYLSWTESLEPLQGAVAK | 141-170 |
| SFQLFGSPPGQR | 304-315 |
| SVDGKEDLIWK | 278-288 |
| SWTESLEPLQGAVAK | 156-170 |
| TESLEPLQGAVAK | 158-170 |
| TVFENLPEK | 232-240 |
| VFENLPEK | 233-240 |
| VLLHQQALFGK | 629-639 |
| VTAIANLK | 685-692 |
| WTESLEPLQGAVAK | 157-170 |
| YAVAVVK | 112-118 |
| YFGYSGAFK | 208-216 |
| YYGYTGAF | 542-549 |
| YYGYTGAFR | 542-550 |

^{#}Residue numbering relates to SEQ ID NO. 1, as described above for Table 1.

TABLE 1b
Peptides present in a peptic hydrolysate
of SEQ ID NO. 2

| Peptide | Residues^{#} |
|---|---|
| AEIYGTKESPQTHY | 98-111 |
| AENRKSSKYSSL | 431-442 |
| AKLGGRPTYE | 668-677 |
| AKLGGRPTYEE | 668-678 |
| AKNLNRED | 580-587 |
| AKNLNREDF | 580-588 |
| AQEKFGKNKSRS | 293-304 |
| ARSVDGKEDL | 276-285 |
| AVVKKANEGLTWNSL | 456-470 |
| DGGMVFEAGRDPYKLRPVA | 79-97 |
| DRDQYEL | 242-248 |
| DRTAGWNIPMGL | 481-492 |
| EAGRDPYKLRPVA | 85-97 |
| EAGRDPYKLRPVAA | 85-98 |
| EAGRDPYKLRPVAAE | 85-99 |
| EIYGTKESPQTHY | 99-111 |
| EKKADAVTL | 70-78 |
| ENLPEKADRDQ | 235-245 |
| ENLPEKADRDQY | 235-246 |
| ENLPEKADRDQYE | 235-247 |
| ENLPEKADRDQYEL | 235-248 |
| ESLEPLQG | 159-166 |
| ESLEPLQGA | 159-167 |
| ESLEPLQGAV | 159-168 |
| FEAGRDPYKLRPVA | 84-97 |
| FEAGRDPYKLRPVAA | 84-98 |
| FGKNKSRS | 297-304 |
| FGSPPGQRDL | 308-317 |
| FGSPPGQRDLL | 308-318 |
| FGSPPGQRDLLF | 308-319 |
| FKCLQDGAGDVAF | 215-227 |
| FKDSALGF | 319-326 |
| FKSETKNLL | 651-659 |
| FNDNTECL | 660-667 |
| FQLFGSPPGQRDLL | 305-318 |
| FRCLAEDVGD | 549-558 |
| GSPPGQRDLL | 309-318 |
| IAEKKADAVT | 68-77 |
| IAEKKADAVTL | 68-78 |
| IPMGI | 146-150 |
| IWKLLSKAQEKFGKNKSRS | 286-384 |
| IWKLLSKAQEKFGKNKSRSFQL | 286-307 |
| IYGTKESPQTHY | 100-111 |
| KAQEKFGKNKSRS | 292-304 |
| KDSALGF | 320-326 |
| KGEADALNL | 405-413 |
| KKADAVTL | 71-78 |
| KSETKNLL | 652-659 |
| KYYGYTGA | 541-548 |
| LECIRA | 62-67 |
| LEGSPPGQRDLL | 307-318 |
| LFKDSALGF | 318-326 |
| LKNLRE | 347-352 |
| LKNLRETAE | 347-355 |
| LNLDGGY | 411-417 |
| LPEKADRDQYE | 237-247 |
| LRIPSKVD | 327-334 |
| LRIPSKVDSA | 327-336 |
| LRIPSKVDSAL | 327-337 |
| LSKAQEKFGKNKSRS | 290-304 |
| LSKAQEKFGKNKSRSFQL | 290-307 |
| LTTLKNLRE | 344-352 |
| LTTLKNLRETAE | 344-355 |
| NLDGGY | 412-417 |
| NLDGGYI | 412-418 |
| NLNREDFRL | 582-590 |
| NLPEKADRDQ | 236-245 |
| NREDFRL | 584-590 |
| PEKADRDQ | 238-245 |
| PEKADRDQYE | 238-247 |
| PEKADRDQYEL | 238-248 |
| PPGQRDLL | 311-318 |
| PYKLRPVA | 90-97 |
| QLFGSPPGQRDLL | 306-318 |
| RSDRAAHVKQVL | 619-630 |
| RSVDGKEDL | 277-285 |
| RTAGWNIPMGL | 482-492 |
| SWTESLEPLQG | 156-166 |
| TESLEPLQG | 158-166 |
| TTLKNLRETAE | 345-355 |
| VARSVDGKEDL | 275-285 |
| VFEAGRDPYKLRPVA | 83-97 |
| VFEAGRDPYKLRPVAA | 83-98 |
| VFEAGRDPYKLRPVAAE | 83-99 |
| VKETTVF | 228-234 |
| VLKGEADAL | 403-411 |
| VSRSDRAAHVKQ | 617-628 |
| VTLDGGM | 76-82 |
| VTLDGGMV | 76-83 |
| VTLDGGMVF | 76-84 |
| VVARSVDGKEDL | 274-285 |
| VVKKANEGLTW | 457-467 |
| VVKKANEGLTWNSL | 457-470 |
| VVSRSDRAAHVKQ | 616-628 |
| VVSRSDRAAHVKQVL | 616-630 |
| WAKNLNRE | 579-586 |
| WAKNLNRED | 579-587 |
| WAKNLNREDF | 579-588 |
| WIIPMGI | 144-150 |
| WNIPMGL | 486-492 |
| YGTKESPQTHY | 101-111 |
| YLGSRY | 338-343 |

^{#}Residue numbering relates to SEQ ID NO. 1, as described above for Table 1.

### Example 2

### Lactoferrin Fragments Inhibit Osteoclast Development

Bone marrow cultures were used to determine the effect of lactoferrin fragments on osteoclast development. The method used has been previously described (see Cornish, et al., (2001)). Bone marrow was obtained from the long bones of four to six week old Swiss male mice by flushing the marrow cavity with media. The cell suspension was incubated for two hours and the non-adherent cells were plated into 48-well plates and cultured in 1.25 vitamin D₃ enriched media for 1 week with lactoferrin fragments at a range of concentrations. Cells were fixed and stained and multinucleated osteoclast-like cells were counted.

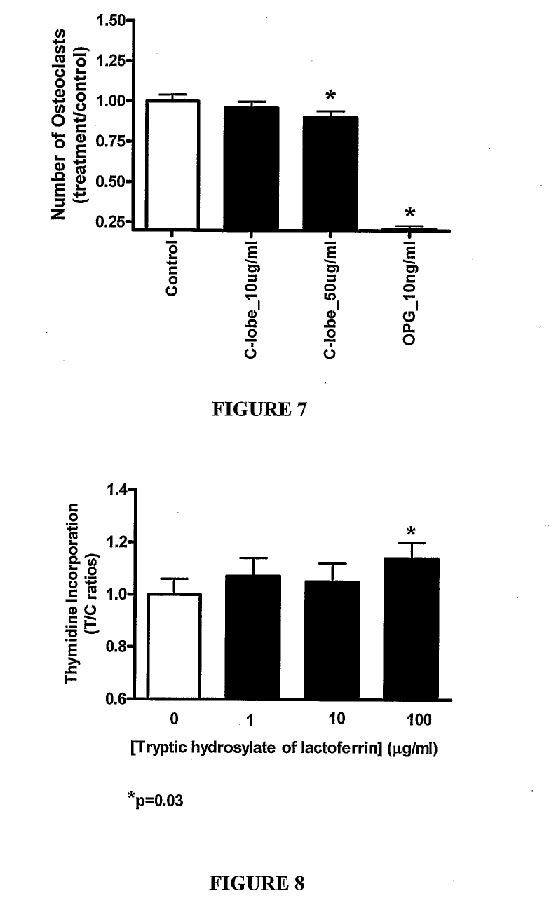

A recombinant N-lobe fragment of full length recombinant human lactoferrin (SEQ ID NO. 5) and a lactoferrin C-lobe fragment (SEQ ID NO. 8) were tested. Osteoprotegerin was used as a positive inhibitor control. Full length recombinant human lactoferrin was also tested. The results are shown in FIGS. 6 and 7, as described above.

### Example 3

### Alternative Peptic Digestion

Bovine lactoferrin (SEQ ID 2) was dissolved to 1 %(w/v) in milliQ water, and the pH adjusted to 2.0 with HCl. Pepsin (Sigma P7012) was added at an E:S of 1:100 and hydrolysis was continued for 20 h at 35° C., with monitoring by SDS-PAGE. The hydrolysis was terminated by adjusting the pH to 8.0 with NaOH, and the hydrolysate heated for 10 min at 80° C. to inactivate enzyme. A small amount of insoluble matter was removed by centrifugation and the supernatant peptides recovered by freeze-drying. Peptides were identified by LC/MS/MS on an Orbitrap ESI-TRAP (Thermo Electron corporation) (Table 1c). The list of peptides does not necessarily exclude other peptides which might have been present but not detected under the analytical conditions.

TABLE 1c
Peptides present in an alternative peptic
hydrolysate of SEQ ID NO. 2

| Peptide | Residues # |
|---|---|
| AEIYGTKESPQTHY | 98-111 |
| AKLGGRPTYE | 668-677 |
| AKLGGRPTYEE | 668-678 |
| AKNLNREDF | 580-588 |
| ARSVDGKEDL | 276-285 |
| AVVKKANEGLTWNSL | 456-470 |
| DGGMVFEAGRDPYKLRPVA | 79-97 |
| DRDQYEL | 242-248 |
| DRTAGWNIPMGL | 481-492 |
| EAGRDPYKLRPVA | 85-97 |
| EAGRDPYKLRPVAA | 85-98 |
| EAGRDPYKLRPVAAE | 85-99 |
| EIYGTKESPQTHY | 99-111 |
| ENLPEKADRDQ | 235-245 |
| ENLPEKADRDQY | 235-246 |
| ENLPEKADRDQYE | 235-247 |
| ENLPEKADRDQYEL | 235-248 |
| ESLEPLQG | 159-166 |
| ESLEPLQGA | 159-167 |
| ESLEPLQGAV | 159-168 |
| FEAGRDPYKLRPVA | 84-97 |
| FGSPPGQRDL | 308-317 |
| FGSPPGQRDLL | 308-318 |
| FGSPPGQRDLLF | 308-319 |
| FKCLQDGAGDVAF | 215-227 |
| FKDSALGF | 319-326 |
| FKSETKNLL | 651-659 |
| FQLFGSPPGQRDLL | 305-318 |
| FRCLAEDVGD | 549-558 |
| IAEKKADAVTL | 68-77 |
| IPMGI | 146-150 |
| IWKLLSKAQEKFGKNKSRSFQL | 286-307 |
| IYGTKESPQTHY | 100-111 |
| KDSALGF | 320-326 |
| KGEADALNL | 405-413 |
| KSETKNLL | 652-659 |
| KYYGYTGA | 541-548 |
| LFGSPPGQRDLL | 307-318 |
| LFKDSALGF | 318-326 |
| LKNLRETAE | 347-355 |
| LRIPSKVDSA | 327-336 |
| LRIPSKVDSAL | 327-337 |
| LSKAQEKFGKNKSRSFQL | 290-307 |
| LTTLKNLRE | 344-352 |
| LTTLKNLRETAE | 344-355 |
| NLDGGYI | 412-418 |
| NREDFRL | 584-590 |
| PEKADRDQ | 238-245 |
| PEKADRDQYE | 238-247 |
| PEKADRDQYEL | 238-248 |
| PPGQRDLL | 311-318 |
| PYKLRPVA | 90-97 |
| QLFGSPPGQRDLL | 306-318 |
| RSVDGKEDL | 277-285 |
| RTAGWNIPMGL | 482-492 |
| SWTESLEPLQG | 156-166 |
| TESLEPLQG | 158-166 |
| VFEAGRDPYKLRPVA | 83-97 |
| VFEAGRDPYKLRPVAA | 83-98 |
| VFEAGRDPYKLRPVAAE | 83-99 |
| VKETTVF | 228-234 |
| VLKGEADAL | 403-411 |
| VTLDGGM | 76-82 |
| [symbol] | 76-83 |
| VTLDGGMVF | 76-84 |
| VVARSVDGKEDL | 274-285 |
| VVKKANEGLTW | 457-467 |
| VVKKANEGLTWNSL | 457-470 |
| WAKNLNRE | 579-586 |
| WAKNLNRED | 579-587 |
| WAKNLNREDF | 579-588 |
| WIIPMGI | 144-150 |
| WNIPMGL | 486-492 |
| YGTKESPQTHY | 101-111 |
| YLGSRY | 338-343 |

# Residue numbering relates to SEQ ID NO. 1, as described above for Table 1. [symbol]
indicates data missing or illegible when filed

### Example 4

### Proliferation of Chondrocytes

Chondrocytes are isolated by removing cartilage (full-depth slices) from the tibial and femoral surfaces of sheep under aseptic conditions. Slices are placed in Dulbecco's Modified Eagles (DME) media containing 5% FBS (v/v) and antibiotics (penicillin 50 g/L, streptomycin 50 g/L and neomycin 100 g/L) and chopped finely with a scalpel blade. Tissue is removed and incubated at 37° C. with firstly pronase (0.8% w/v for 90 minutes) followed by collagenase (0.1% w/v for 18 hours) to complete the digestion. Cells are isolated from the digest by centrifugation (10 minutes at 1300 rpm), resuspended in DME/5% FBS, passed through a nylon mesh screen of 90 Fm pore size to remove any undigested fragments, and re-centrifuged. The cells are then washed and resuspended twice in the same media, seeded into a 75 cm² flask containing DME/10% FBS, and incubated under 5% CO₂/95% air at 37° C. Confluence is reached by 7 days, at which time the cells are subcultured. After trypsinization using trypsin-EDTA (0.05%/0.53 mM), the cells are rinsed in DME/5% FBS and resuspended in a fresh medium, then seeded into 24-well plates (5×104 cells/mL, 0.5 mL/well). Measurement of thymidine incorporation is performed in growth-arrested cell populations as for the osteoblast-like cell cultures described above.

### Example 5

### Stimulation of Bone Growth In Vivo

The mouse model described by Cornish, et al. ((1993) Endocrinology 132, 1359-1366) may be used to assess the stimulation of bone growth in vivo by lactoferrin fragments and hydrolysates. Injections of lactoferrin fragments or hydrolysates are given daily for 5 days, and the animals sacrificed a week later. Bone formation is determined by fluorescent labelling of newly formed bone. Indices of bone resorption and of bone mass are determined by conventional light microscopy, assisted by image analysis software.

### APPLICATIONS

### Application 1

Set yoghurts of between 14 and 17% solids, with or without fruit added, can be prepared as follows. Medium heat skim milk powder (between 109-152 g) and ALACO stabilizer (100 g) are reconstituted with approximately 880 ml of 50° C. water. Anhydrous Milk Fat (20 g) is then added and mixed for 30 min. The mixture is then heated to 60° C., homogenized at 200 bar, and then pasteurized at 90° C. After cooling to a temperature between 40-42° C., a starter mixture and the freeze-dried protein preparation described above (up to 50 mg of a lactoferrin fragment or hydrolysate or mixture thereof at 95% purity or an equivalent quantity from a not so highly purified source) is added. If desired, fresh fruit may also be added at this point. The mixture is then filled into containers, incubated at 40° C. until pH 4.2-4.4 is reached, and then chilled in a blast cooler.

An alternative method for preparing the same set yoghurts is by dry blending the indicated quantity of lactoferrin fragment or hydrolysate or mixture thereof or the indicated quantity as a dose rate, into the dry milk solids, prior to its use in the yoghurt formulation.

### Application 2

Dry blends of either skim or whole milk powder with calcium and the freeze dried lactoferrin fragment or hydrolysate or mixture thereof preparations can give dairy based formulations or compositions which can be used either as functional foods or as functional food ingredients. Such compositions can be used as reconstituted milks, milk powder ingredients, dairy desserts, functional foods, cheeses or butter or beverages, and nutraceuticals or dietary supplements. Blending the dry ingredients in ratios of milk powder:calcium:active lactoferrin fragment or hydrolysate between 90:9.5:0.5 and 94:5.95:0.0001 provide compositions suitable for such uses.

### Application 3

Blended compositions of milk powder, calcium, and the lactoferrin fragment or hydrolysate or mixture thereof can be used as bone health functional foods, bone health food ingredients, or as a food ingredient for delivery of bone health nutrients in a range of health foods.

For such compositions, the calcium and protein contents of the compositions need to be adjusted to required, allowable nutritional limits. Commercially available ingredient milk powders typically contains between 300 and 900 mg calcium per 100 g powder, depending upon their sources. A source of calcium may be added to the powder to extend the calcium content up to 3% by weight of the ingredient milk powder as a blend. The protein level of commercially available ingredient milk or dairy-based protein powders varies depending upon the type of the ingredient, the method of its manufacture, and its intended use. Ingredient milk powder typically contains between 12% and 92% protein. Examples are commercially available skim and whole milk powders, food grade caseins, caseinates, milk protein concentrate powders, spray dried ultrafiltered or microfiltered retentate powders, and the milk protein isolate products. The lactoferrin fragment or hydrolysate or mixture thereof may be incorporated into a protein and calcium blend to give nutritional milk powders that can be used as ingredients in healthy foods and drinks. Such blends provide ingredients suitable for use in preparing yoghurts and yoghurt drinks, acid beverages, ingredient milk powder blends, pasteurized liquid milk products, UHT milk products, cultured milk products, acidified milk drinks, milk-and-cereal combination products, malted milks, milk-and-soy combination products. For such uses, the blend can have a composition where the calcium content is between 0.001% and 3.5% (w/w), the protein composition is between 2% and 92%, and lactoferrin fragment or hydrolysate or mixture thereof as the osteoblast proliferating agent is added at levels between 0.000001% and 5.5%.

### INDUSTRIAL APPLICATION

The medicinal uses and methods of the present invention may be used for stimulating skeletal growth, inhibiting bone resorption, stimulating chondrocyte proliferation, stimulating osteoblast proliferation, inhibiting osteoclast development or treating or preventing a skeletal, joint or cartilage disorder. The uses and methods may be carried out employing dietary (as foods or food supplements), nutraceutical or pharmaceutical compositions comprising at least one lactoferrin fragment or lactoferrin hydrolysate or a mixture thereof.

Those persons skilled in the art will understand that the above description is provided by way of illustration only and that the invention is not limited thereto.

TABLE 2
Lactoferrin fragments and hydrolysates

| Lactoferrin Peptides | Residues | Manufacture/cleavage |
|---|---|---|
| Bovine lactoferricin | | Trypsin digest |
| (from SEQ ID NO. 2) | | |
| Bovine lactoferrin hydrolysate | Total hydrolysate | Pepsin |
| (from SEQ ID NO. 2) | (unspecified) | |
| Bovine lactoferrin hydrolysate | Total hydrolysate | Pepsin |
| (from SEQ ID NO. 2) | (unspecified) | |
| Bovine lactoferricin | r17-41 | In vivo gastric digestion (10 min) |
| (and larger fragments) | r17-42 | of 2 g (200 mL of 10 mg/mL) |
| (from SEQ ID NO. 2) | r17-43 | b lactoferrin orally fed to adult |
| | r17-44 | human (starved 12 h) |
| | r12-44 | |
| | r9-58 | |
| | r16-79 | |
| | r13-36 | |
| Bovine N-terminal fragment | r17-42 | Pepsin |
| (from SEQ ID NO. 2) | r1-16/43-48 (disulfide linked) | |
| | r1-48 (cleaved 42-43, disulfide | |
| | linked) | |
| Bovine lactoferricin | | |
| (from SEQ ID NO. 2) | | |
| Bovine lactoferricin | | Pepsin |
| (from SEQ ID NO. 2) | | |
| Bovine lactoferricin | | |
| (from SEQ ID NO. 2) | | |
| Lactoferricin 15 residue derivatives | Equivalent to r17-31 of | Synthetic |
| bovine, human, caprine, murine | bovine lactoferricin | |
| & porcine. | (from SEQ ID NO. 2) | |
| and | Equivalent to r17-31 of | Synthetic |
| modified human, caprine, | bovine lactoferricin | |
| porcine | (from SEQ ID NO. 2) | |
| Bovine lactoferricin and derivatives | r17-41 | Recombinant chymosin |
| (from SEQ ID NO. 2) | subfragment 1-10 (r17-26) | Recombinant chymosin |
| | subfragment 11-26 (r27-41) | Recombinant chymosin |
| Bovine lactoferricin | | Pepsin |
| (from SEQ ID NO. 2) | | |
| Bovine lactoferrin hydrolysates | Total hydrolysate | Porcine & cod pepsin, |
| (from SEQ ID NO. 2) | Total hydrolysate | Penicillum duponti acid protease |
| | Low MW peptides | Porcine pepsin |
| Bovine lactoferrin hydrolysate | | |
| (from SEQ ID NO. 2) | | |
| Bovine lactoferrin peptides | r324-329 (YLTTLK) | Trypsin |
| (from SEQ ID NO. 2) | r324-329 (YLTTLK) | Synthetic |
| | r86-258 (ESPQT . . . AVVAR) | Trypsin |
| Bovine lactoferricin | | |
| (from SEQ ID NO. 2) | | |
| Bovine lactoferricin | | Pepsin |
| (from SEQ ID NO. 2) | | |
| Bovine lactoferricin | | |
| (from SEQ ID NO. 2) | | |
| Bovine lactoferricin | | |
| (from SEQ ID NO. 2) | | |
| Bovine lactoferricin | | |
| (from SEQ ID NO. 2) | | |
| Bovine lactoferrin fragments | r17-42 (17FKCRR . . . RRAFA42) | In vivo digestion (adult rat) |
| (from SEQ ID NO. 2) | Masses 42, 36,, 33 & 29 kDa | |
| | detected | |
| Bovine lactoferrin hydrolysate | | Pepsin |
| & lactoferricin | | |
| (from SEQ ID NO. 2) | | |
| Bovine lactoferrin | | In vivo digestion, human infants |
| (from SEQ ID NO. 2) | | |
| Bovine lactoferrin hydrolysate | Total hydrolysate | Pepsin |
| (from SEQ ID NO. 2) | | |
| Human (from SEQ ID NO. 4) & | | In vivo digestion, infant rhesus |
| bovine (from SEQ ID NO. 2) milk | | monkeys preterm, 6 weeks |
| lactoferrin | | & 7 months |
| Bovine lactoferricin | | Pepsin |
| (from SEQ ID NO. 2) | | |
| Bovine lactoferrin peptides | | Trypsin |
| (from SEQ ID NO. 2) | | |
| Bovine lactoferrin N/C lobes | | Trypsin |
| (from SEQ ID NO. 2) | | |
| Bovine lactoferrin hydrolysates | Total | Pepsin, trypsin |
| (from SEQ ID NO. 2) | plus | |
| | r17-38 | Pepsin |
| | r1-16/45-48 (disulfide linked) | Pepsin & synthetic |
| | r1-15/45-46 (disulfide linked) | Pepsin & synthetic |
| | r1-13 | Pepsin & synthetic |
| Bovine lactoferricin peptide | r20-25 | |
| (from SEQ ID NO. 2) | (RRWQWR) | |
| Bovine lactoferrin hydrolysate & | | Pepsin |
| Bovine lactoferrin lactoferricin | | Pepsin |
| (from SEQ ID NO. 2) | | |
| Bovine lactoferricins | r17-41 | Pepsin |
| (from SEQ ID NO. 2) | r17-31 | Synthetic |
| | r17-31 (D-amino acids) | Synthetic |
| Bovine lactoferrin | | Gastric pepsin in vivo |
| (from SEQ ID NO. 2) | | |
| Bovine lactoferricin | | Pepsin |
| (from SEQ ID NO. 2) | | |
| Bovine lactoferricin | | Human gastric pepsin cleavage |
| (from SEQ ID NO. 2) | | |
| Human lactoferricin | | Human gastric pepsin cleavage |
| (from SEQ ID NO. 4) | | |
| Bovine lactoferrin | | Trypsin/chymotrypsin |
| (from SEQ ID NO. 2) | | |
| Bovine lactoferrin-apo form | | Trypsin |
| (from SEQ ID NO. 2) | | |
| Bovine lactoferrin-holo form | | Trypsin |
| (from SEQ ID NO. 2) | | |
| Bovine lactoferricin | | Pepsin |
| (from SEQ ID NO. 2) | | |
| Bovine lactoferrin | | Chymosin |
| (from SEQ ID NO. 2) | | Plasmin |
| Human lactoferrin | | Chymosin |
| (from SEQ ID NO. 4) | | Plasmin |
| Bovine lactoferrin digest | | Pepsin |
| (from SEQ ID NO. 2) | | |
| Bovine lactoferrin | | Pepsin digest |
| (from SEQ ID NO. 2) | | |
| Bovine lactoferrampin | r268-284 | Synthetic |
| (from SEQ ID NO. 2) | 268WKLLSKAQEKFGKNKSR284 | |
| Bovine lactoferrampin peptides | r268-278 | Synthetic |
| (from SEQ ID NO. 2) | WKLLSKAQEKF | |
| | r279-284 | Synthetic |
| | GKNKSR | |
| Bovine lactoferrin peptides | Collectively a low ConA Lf fraction | Found in a low ConA affinity Lf |
| (from SEQ ID NO. 2) | 56 kDa (r329 NLRETAEEVKA . . . ) | fraction from mastitic mammary |
| | 38 kDa (r1 APRKNVRWCTIS . . . ) | gland secretion. |
| | 23 kDa (r237 APVDAFKECHLA . . . ) | |
| | 22 kDa (r285 SFQFLGSPPGO . . . ) | |
| | 19 kDa (r240 RYTRVVWCAVG . . . ) | |
| Bovine lactoferricin peptide | RRWQWR-NH2 | Synthetic amidated |
| (from SEQ ID NO. 2) | | |
| Bovine lactoferricin peptide | RRWQWRMKKLG | Synthetic |
| (from SEQ ID NO. 2) | | |
| Bovine lactoferricin peptide | RRWQWRMKKLG | Synthetic, linear |
| (from SEQ ID NO. 2) | (LfcinB4-14) | |
| | RRWQWRMKKLG-NH2 | Synthetic, amidated |
| | (LfcinB4-14-NH2) | |
| | CRRWQWRMKKLGC-NH2 | Synthetic, disulfide bonded |
| | (LfcinB4-14Disu) | on flanking cys residues, cyclic, |
| | | amidated to incr cationic nature |
| Bovine lactoferricin peptide | r153-183, human homologue | Synthetic |
| (kaliocin-1) | 153FFSASCVPGADKGQQFPNL- | |
| (from SEQ ID NO. 2) | CRLCAGTGENKCA183 (31 aa) | |
| Human Lactoferricin-type peptide | r 17-39 (TKCFQWQRNMRKVR- | Synthetic |
| (Lfpep) (from SEQ ID NO. 4) | GPPVSCIKR (23aa) | |
| Human Lactoferricin-type peptide | r 17-39 (TKCFQWQRNMRKVR- | Synthetic |
| (Lfpep) (from SEQ ID NO. 4) | GPPVSCIKR (23aa) | |
| Human lactoferrin-large fragments | r3-691, 78 kDa, | In vivo proteolysis (trypsin-like) |
| (from SEQ ID NO. 4) | 3RRRSVQWC . . . FLRK691; | of maternal Lf in preterm infants |
| | r3-283 (N-lobe fragment) 39 kDa, | fed human milk |
| | 3RRRSVQWC . . . FGKDK283; | |
| | r284-691 (C-lobe fragment), | |
| | 51 kDa, 284SPKFQLFGSP . . . FLRK691 | |
| Human lactoferrin-‘half-lactoferrins’ | 2 × 40 kDa fragments | Cleavage of human lactoferrin at |
| (from SEQ ID NO. 4) | | pH4, 100 C for 2 min |
| Bovine lactoferrin large fragments | 51 kDa, 285SFQLFGSP . . . R689 | Trypsin |
| (from SEQ ID NO. 2) | 44 kDa, 389ARYTR . . . R689 | |
| | 36 kDa, 1APRKNVRW . . . R284 | |
| Human lactoferrin large fragments | C-terminal fragment | Pepsin digestion, pH 3.0, |
| (from SEQ ID NO. 4) | (~41-42 kDa) | of iron-saturated human Lf |
| | C- & N-terminal fragment | Trypsin |
| | (~40 kDa & ~36 kDa respectively) | |
| | N-terminal (~46 kDa) & C-terminal | Chymotrypsin |
| | fragment (~40 kDa) | |
| Human lactoferrin large fragments | N-terminal fragment r3-281, 30 kDa | Trypsin cleavage of diferric Lf |
| (from SEQ ID NO. 4) | C-term fragment r282-703, 50 kDa | |
| | C-term peptic fragment r339-703 | Pepsin |
| Human lactoferrin ‘N2- | r 91-257, 18.5 kDa | Trypsin digest of N-tryptic |
| glycopeptide’ | | fragment |
| (from SEQ ID NO. 4) | | r3-281 |
| Bovine lactoferricin containing | | In vivo hydrolysis in mice fed |
| peptides | | commercial milk enriched with |
| (from SEQ ID NO. 2) | | 40 mg/mL lactoferrin |
| Amino-peptidase modified human | r15-691 | Aminopeptidase M |
| lactoferrin (from SEQ ID NO. 4) | | |
| Bovine lactoferrin large fragment | ~30-35 kDa | Isolated from endothelial cells |
| (from SEQ ID NO. 2) | | and mononuclear phagocytes as |
| | | predominant form of Lf |
| Bovine lactoferrin large fragment | ~35 kDa, r357... | Thermolysin digest of bovine Lf |
| C-terminal (from SEQ ID NO. 2) | (VKARYRVVWXAVGGP . . . ) | |
| Human lactoferrin large peptides | N-tryptic fragment (N-lobe), r4-281 | Mild tryptic hydrolysis |
| 30, 50 kDa fragments | C-tryptic fragment (C-lobe), r282-703 | Mild tryptic hydrolysis |
| (from SEQ ID NO. 4) | N/C-tryptic combined r91-257 (N2-glycopeptide) | Added together |
| | | Tryptic hydrolysis of the 30 kDa |
| | | frag |
| Human lactoferricin peptides | r20-35 (FQWQRNMRKVRGPPVS | Synthetic |
| (from SEQ ID NO. 4) | (loop region) | |
| | 2 peptides from r20-35 | Cyanogen bromide cleavage of |
| | (+ve charge and aromatic terminus) | r20-35 |
| | r24-35 (FQWQRNMRKVR) | Synthetic |
| | (charged portion of loop) | |
| | r31-35 (GPPVS) | Synthetic |
| | (uncharged loop region) | |
| Bovine lactoferricin | r17-41 | Pepsin |
| (from SEQ ID NO. 2) | | |
| Human lactoferricin | r18-40 | Synthetic |
| (from SEQ ID NO. 4) | | |
| Bovine lactoferricin | | Pepsin |
| (from SEQ ID NO. 2) | | |
| Human lactoferricin peptides | r20-35 (FQWQRNMRKVRGPPVS) | Synthetic (=HLP 1) |
| (from SEQ ID NO. 4) | (loop region) | |
| | r20-30 (FQWQRNMRKVR) | Synthetic (=HLP 2) |
| | (alpha-helical loop region) | |
| | r20-30 (FQWQRNPRKVR) | Synthetic (=HLP 6) |
| | (alpha-helical loop region) | Proline substituted for |
| | | methionine |
| | r20-30 (FQWQRNMRKVR) | Synthetic (=HLP 7) |
| | (a-hlx loop region, D amino-acids) | All D-amino acids |
| Human lactoferricin | 49 residues (r1-49) | Pepsin |
| (from SEQ ID NO. 4) | Gly1 . . . Ala49 | |

| Lactoferrin Peptides | References |
|---|---|
| Bovine lactoferricin | Eliassen, et al., (2002) Anticancer Research |
| (from SEQ ID NO. 2) | 22(5) 2703-10 |
| Bovine lactoferrin hydrolysate | Murdock & Matthews (2002) J Applied |
| (from SEQ ID NO. 2) | Microbiology 93 (5) 850-6 |
| Bovine lactoferrin hydrolysate | Tomita et al., (2002) Biochemistry & Cell |
| (from SEQ ID NO. 2) | Biology 80 (1) 109-12 |
| Bovine lactoferricin | Kuwata et al., (1998) Advances in Exptl |
| (and larger fragments) | Medicine & Biology 443, 23-32 |
| (from SEQ ID NO. 2) | Kuwata et al., (1998) Biochim Biophys Acta |
| | 1429 (1) 129-41 |
| Bovine N-terminal fragment | Dionysius & Milne (1997) J. Dairy Science 80 |
| (from SEQ ID NO. 2) | (4) 667-74 |
| Bovine lactoferricin | Bellamy et al., (1993) J. Applied Bacteriology |
| (from SEQ ID NO. 2) | 75 (5) 478-84 |
| Bovine lactoferricin | Bellamy et al., (1994) Letters in Applied |
| (from SEQ ID NO. 2) | Microbiology 18 (4) 230-233 |
| Bovine lactoferricin | Bellamy et al., (1993) Medical Microbiology |
| (from SEQ ID NO. 2) | and Immunology 182 (2) 97-105 |
| Lactoferricin 15 residue derivatives | Strom et al., (2000) Journal of Peptide |
| bovine, human, caprine, murine | Research 56 (5) 265-274 |
| & porcine. | |
| and | |
| modified human, caprine, | |
| porcine | |
| Bovine lactoferricin and derivatives | Hoek et al., (1997) Antimicrobial Agents |
| (from SEQ ID NO. 2) | and Chemotherapy 41 (1) 54-59 |
| Bovine lactoferricin | Jones et al., (1994) J Applied Bacteriology |
| (from SEQ ID NO. 2) | 77 (2) 208-14 |
| Bovine lactoferrin hydrolysates | Tomita et al., (1991) J Dairy Science |
| (from SEQ ID NO. 2) | 74 (12) 137-142 |
| Bovine lactoferrin hydrolysate | Rahman et al., (2004) Miruku Saiensu |
| (from SEQ ID NO. 2) | 53 (4) 325-27 |
| Bovine lactoferrin peptides | Superti et al., (2001) Biochim Biophys Acta |
| (from SEQ ID NO. 2) | 1528 (2-30) 107-15 |
| Bovine lactoferricin | Longhi et al., (2005) Int J Immunopathology |
| (from SEQ ID NO. 2) | & Pharmacology 18 (2) 317-25 |
| Bovine lactoferricin | Yoo et al., (1997) Biochem. Biophys. Res. |
| (from SEQ ID NO. 2) | Comm. 237 (3) 624-8 |
| Bovine lactoferricin | Yoo et al., (2000) Lactoferrin: structure, |
| (from SEQ ID NO. 2) | function and applications. Proc 4th Int Conf |
| | on Lactoferrin, Sapporo, Japan, 18-22 May |
| | 1999, p163-171 |
| Bovine lactoferricin | McCann et al., (2003) J. Applied Microbiology |
| (from SEQ ID NO. 2) | 95 (5) 1026-33 |
| Bovine lactoferricin | Berkhout et al., (2002) Antiviral Research |
| (from SEQ ID NO. 2) | 55 (2) 341-55 |
| Bovine lactoferrin fragments | Kuwata et al., (2001) J Nutrition 131 (8) |
| (from SEQ ID NO. 2) | 2121-7 |
| Bovine lactoferrin hydrolysate | Masschalck et al., (2001) Int J Food |
| & lactoferricin | Microbiology 64 (3) 325-32 |
| (from SEQ ID NO. 2) | |
| Bovine lactoferrin | Spik et al., (1982) Acta Paediatrica Scand |
| (from SEQ ID NO. 2) | 71, (6)979-985 |
| Bovine lactoferrin hydrolysate | Miyauchi et al., (1997) 80 (10) |
| (from SEQ ID NO. 2) | 2330-9 |
| Human (from SEQ ID NO. 4) & | Lindberg et al., (1997) J. Pediatric Gastro- |
| bovine (from SEQ ID NO. 2) milk | enterology and Nutrition 24 (5) 537-43 |
| lactoferrin | |
| Bovine lactoferricin | Longhi et al., (1994) Medical Microbiology |
| (from SEQ ID NO. 2) | and Immunology 183 (2) 77-85 |
| Bovine lactoferrin peptides | Siciliano et al., (1999) Biochem. Biophys. |
| (from SEQ ID NO. 2) | Res. Comm. 264 (1) 19-23 |
| Bovine lactoferrin N/C lobes | Shimazaki et al., (1994) Int Dairy Fed, Proc |
| (from SEQ ID NO. 2) | seminar Uppsala, 1993, p122-130 |
| Bovine lactoferrin hydrolysates | Roy et al., (2002) J Dairy Science 85 (9) |
| (from SEQ ID NO. 2) | 2065-2074 |
| Bovine lactoferricin peptide | Schibli et al., (1999) FEBS Letters 446 (2-3) |
| (from SEQ ID NO. 2) | 213-217 |
| Bovine lactoferrin hydrolysate & | Shin, et al., (1998) Letters in |
| Bovine lactoferrin lactoferricin | Appl. Microbiology 26 (6) 407-411 |
| (from SEQ ID NO. 2) | |
| Bovine lactoferricins | Ulvatne & Vorland (2001) Scand. J. Infect. |
| (from SEQ ID NO. 2) | Diseases 33 (7) 507-11 |
| Bovine lactoferrin | Troost et al., (2001) J Nutrition 131 (8) 2101-4 |
| (from SEQ ID NO. 2) | |
| Bovine lactoferricin | Bellamy et al., (1992) J Applied Bacteriology |
| (from SEQ ID NO. 2) | 73 (6) 472-9 |
| Bovine lactoferricin | Bellamy et al., (1992) Biochim Biophys Acta |
| (from SEQ ID NO. 2) | 1121 (1-2) 130-6 |
| Human lactoferricin | |
| (from SEQ ID NO. 4) | |
| Bovine lactoferrin | Brines & Brock (1983) Biochim Biophys Acta |
| (from SEQ ID NO. 2) | 759 (3) 229-35 |
| Bovine lactoferrin-apo form | Brock et al., (1976) Biochim Biophys Acta |
| (from SEQ ID NO. 2) | 446 (1) 214-25 |
| Bovine lactoferrin-holo form | El-sayad et al., (2003) |
| (from SEQ ID NO. 2) | |
| Bovine lactoferricin | Milchwissenschaft 58 (5/6) 266-270 |
| (from SEQ ID NO. 2) | |
| Bovine lactoferrin | Shimazaki et al., (1991) Agricultural & |
| (from SEQ ID NO. 2) | Biological Chemistry 55 (4) 1125-6 |
| Human lactoferrin | |
| (from SEQ ID NO. 4) | |
| Bovine lactoferrin digest | Facon & Skura (1996) Int Dairy Journal |
| (from SEQ ID NO. 2) | 6 (3) 303-13 |
| Bovine lactoferrin | Sakai et al., (2004) Miriku Saiensu 53 (4) |
| (from SEQ ID NO. 2) | 254-255 |
| Bovine lactoferrampin | van der Kraan et al., (2004) Peptides, 177-183 |
| (from SEQ ID NO. 2) | |
| Bovine lactoferrampin peptides | van der Kraan et al., (2004) Peptides, 177-183 |
| (from SEQ ID NO. 2) | |
| Bovine lactoferrin peptides | Komine et al., (2005) J. Vet. Med. Sci. 67 (7) |
| (from SEQ ID NO. 2) | 667-677 |
| Bovine lactoferricin peptide | Tomita et al., (1994) Acta Paediatr. Jpn 36, |
| (from SEQ ID NO. 2) | 585-591 |
| Bovine lactoferricin peptide | Kang et al., (1996) Int. J. Pept. Protein Res. |
| (from SEQ ID NO. 2) | 48, 357-363 |
| Bovine lactoferricin peptide | Nguyen et al., (2005) J. Peptide Sci 11, |
| (from SEQ ID NO. 2) | 379-389 |
| Bovine lactoferricin peptide | Viejo-Diaz et al., (2003) Biochemistry |
| (kaliocin-1) | (Moscow) 68 (2) 217-227 |
| (from SEQ ID NO. 2) | |
| Human Lactoferricin-type peptide | Viejo-Diaz et al., (2003) Biochemistry |
| (Lfpep) (from SEQ ID NO. 4) | (Moscow) 68 (2) 217-227 |
| Human Lactoferricin-type peptide | Aguilera et al., (1999) FEBS Letters 462, (3) |
| (Lfpep) (from SEQ ID NO. 4) | 273-277 |
| Human lactoferrin-large fragments | Hutchens et al., (1991) Proc. Natl. Acad. Sci. |
| (from SEQ ID NO. 4) | USA 88, 2994-2998 |
| Human lactoferrin-‘half-lactoferrins’ | Davidson & Lonnerdal (1989) Am. J. Physiol. |
| (from SEQ ID NO. 4) | 257, (6) G930-G934. |
| Bovine lactoferrin large fragments | Sitaram & McAbee (1997) Biochem J. |
| (from SEQ ID NO. 2) | 323, 815-822 |
| Human lactoferrin large fragments | Bluard-Deconinck et al., (1978) Biochem. J. |
| (from SEQ ID NO. 4) | 171, 321-327 |
| Human lactoferrin large fragments | Legrand et al., (1986) Biochem. J. 236, 839-844 |
| (from SEQ ID NO. 4) | |
| Human lactoferrin ‘N2- | Legrand et al., (1986) Biochem. J. 236, 839-844 |
| glycopeptide’ | |
| (from SEQ ID NO. 4) | |
| Bovine lactoferricin containing | Kuwata e al., (1998) Biochem. J 334 (2) |
| peptides | 321-323 |
| (from SEQ ID NO. 2) | |
| Amino-peptidase modified human | Ziere et al., (1996) Biochem. J. 313, 289-295 |
| lactoferrin (from SEQ ID NO. 4) | |
| Bovine lactoferrin large fragment | Schmidt et al., (1993) J. Clin. Investigation |
| (from SEQ ID NO. 2) | 92, 2155-2168 |
| Bovine lactoferrin large fragment | Schmidt et al., (1994) J. Biol. Chem. 13, |
| C-terminal (from SEQ ID NO. 2) | 9882-9888 |
| Human lactoferrin large peptides | Rochard et al., (1989) FEBS Lett. 255 (1) |
| 30, 50 kDa fragments | 201-204 |
| (from SEQ ID NO. 4) | |
| Human lactoferricin peptides | Odell et al., (1996) FEBS Letters 382, (1/2) 175-178 |
| (from SEQ ID NO. 4) | |
| Bovine lactoferricin | Turchany et al., (1995) Infection & Immunity |
| (from SEQ ID NO. 2) | 63 (11) 4550-4552 |
| Human lactoferricin | |
| (from SEQ ID NO. 4) | |
| Bovine lactoferricin | Yamauchi et al., (1993) Infection & Immunity 61 |
| (from SEQ ID NO. 2) | (2) 719-728 |
| Human lactoferricin peptides | Chapple et al., (1998) Infection & Immunity 66 |
| (from SEQ ID NO. 4) | (6) 2434-2440 |
| Human lactoferricin | Hunter et al., (2005) Antimicrobial Agents & |
| (from SEQ ID NO. 4) | Chemotherapy |

### REFERENCES

Aguilera O, Ostolaza H, Quirós L M, Fierro J F. Permeabilizing action of an antimicrobial lactoferricin-derived peptide on bacterial and artificial membranes. FEBS Lett. 1999, 462 (3) 273-277.
Ainscough, E W, Brodie A, M.; Plowman J E. The chromium, manganese, cobalt and copper complexes of human lactoferrin. Inorganica Chimica Acta 1979,33 (2) 149-53.
Baker E N, Baker H M, Kidd R D., Lactoferrin and transferrin: functional variations on a common structural framework. Biochem Cell Biol. (2002) 80(l):27-34.
Bellamy W, Yamauchi K, Wakabayashi H, Takase M, Takakura N, Shimamura S, Tomita M. Antifungal properties of lactoferricin B, a peptide derived from the N-terminal region of bovine lactoferrin. Left. In Applied Microbiology 1994, 18 (4) 230-233.
Bellamy W R, Wakabayashi H, Takase M, Kawase K, Shimamuru S, Tomita M. Role of cell binding in antibacterial mechanism of lactoferricin B. J. Applied Bacteriology 1993, 75 (5) 478-84.
Bellamy W R, Wakabayashi H, Takase M, Kawase K, Shimamuru S, Tomita M. Killing of Candida albicans by lactoferricin B, a potent antimicrobial peptide derived from the N-terminal region of bovine lactoferrin. Medical Microbiolgy and Immunology 1993, 182 (2) 97-105.
Bellamy W R, Takase M, Wakabayashi H, Kawase K, Tomita M. Antibacterial spectrum of lactoferricin B, a potent bactericidal peptide derived from the N-terminal region of bovine lactoferrin. J. Applied Bacteriology 1992, 73 (6) 472-9.
Bellamy W, Takase M, Yamauchi K, Wakabayashi H, Kawase K, Tomita M. Identification of the bactericidal domain of lactoferrin. Biochim Biophys Acta. (1992) 1121(1-2): 130-6.
Bluard-Deconinck J M, Williams J, Evans R W, van Snick J, Osinski P A, Masson P L. Iron-binding fragments from the N-terminal and C-terminal regions of human lactoferrin. Biochemical Journal 1978, 171 (2) p321-7.
Bowie J U, Reidhaar-Olson J F, Lim W A, Sauer R T. Deciphering the message in protein sequences: tolerance to amino acid substitutions. Science. (1990) 247(4948):1306-10.
Brines R D, Brock J H. The effect of trypsin and chymotrypsin on the in vitro antimicrobial and iron-binding properties of lactoferrin in human milk and bovine colostrum. Unusual resistance of human apolactoferrin to proteolytic digestion. Biochim. Biophys Acta 1983, 759 (3) 229-35.
Brock J H, Arzabe F, Lampreave F, Pineiro A. The effect of trypsin on bovine transferrin and lactoferrin. Biochim. Biophys. Acta 1976, 446 (1) 214-25.Cadwell, R. C. and G. F. Joyce. Randomization of genes by PCR mutagenesis. PCR Methods Appl. (1992) 2: 28-33.
Cadwell, R. C. and G. F. Joyce. Randomization of genes by PCR mutagenesis. PCR Methods Appl. 1992; 2: 28-33.
Chapple D S, Mason D J, Joannou C L, Odell E W, Gant V, Evans R W. Structure-function relationship of antibacterial syntheic peptides homologous to a helical surface region on human lactoferrin against Escherichia coli Serotype O111. Infect. Immun. 1998, 66 (6) 2434-40.
Cornish J, Callon I E, Bava U, Kamona S A, Cooper G J, Reid I R. Effects of calcitonin, amylin, and calcitonin gene-related peptide on osteoclast development. Bone. (2001) 29(2): 162-8.
Davidson L A, Lonnerdal B. Fe-saturation and proteolysis of human lactoferrin: effect on brush-border receptor-mediated uptake of Fe and Mn. Am J Physiol Gastrointest Liver Physiol. 1989, 257 (6) 930-34.
Dionysius D A, Milne J M. Antibacterial peptides of bovine lactoferrin: purification and characterization. Journal of Dairy Science 1997, 80 (4) 667-7.
Eliassen L T, Berge G, Sveinbjomsson B, Svendsen J S, Vorland L H, Rekdal. Evidence for a direct antitumor mechanism of action of bovine lactoferricin. Anticancer Research (2002) 22 (5) 2703-10.
El-Sayed E M, Mahfouz M B, Abdelsalam A M, El-Gawad I A A, Naim H Y. Antimicrobial activity of recombinant bovine lactoferrin and lactoferricin B and glycosylated human lysozyme. Milchwissenschaft 2003, 58 (5/6) 266-270.
Facon M J, Skura B J. Antibacterial activity of lactoferricin, lysozyme and EDTA against Salmonella enteritidis. Int. Dairy J. 1996, 6 (3) 303-13.
Foley A A, Bates G W. The purification of lactoferrin from human whey to batch extraction Analytica lBiochemistry 1987, 162 (1) 296-300.
Freireich E J, Gehan E A, Rall D P, Schmidt L H, Skipper H E., Quantitative comparison of toxicity of anticancer agents in mouse, rat, hamster, dog, monkey, and man. Cancer Chemother Rep. (1966) 50(4):219-44.
Groot C G, Danes J K, van der Meer J M, Herrmann-Erlee M P, Osteocalcin antigenicity in cultured osteoblast-like cells after stimulation with 1,25-vitamin D3. Cell Biol Int Rep. (1985) 9(6):528.
Hermann-Erlee M, van der Meer J., The effect of PTH and PGE2 on growth and differentiation of primary fetal rat osteoblast-like cells, on UMR106 osteosarcoma cells, and on a SV40 transformed ‘osteoblast-like’ cell line. 9th International Conference of Calcium Regulatory Hormones and Bone Metabolism, Montréal, Canada, 1986, p 409.
Hoek K S, Milne J M, Grieve P A, Dionysius D A, Smith R; Anti-bacterial activity of bovine lactoferrin-derived peptides. Antimicrobial Agents and Chemotherapy 1997, 41 (1) 54-59.
Hunter H N, Demcoe A R, Jenssen H, Gutteberg T J, Vogel H J. Human Lactoferricin Is Partially Folded in Aqueous Solution and Is Better Stabilized in a Membrane Mimetic Solvent. Antimicrob. Agents Chemother. 2005, 49 (8) 3387-95
Hutchens T W, Henry J F, Yip T. Structurally Intact (78-kDa) Forms of Maternal Lactoferrin Purified from Urine of Preterm Infants Fed Human Milk: Identification of a Trypsin-Like Proteolytic Cleavage Event In vivo that does not Result in Fragment Dissociation. Proc. Natl. Acad. Sci. 1991, 88 (8) 2994-8.
Hwang P M, Zhou N, Shan X, Arrowsmith C H, Vogel H J. Three-dimensional solution structure of lactoferricin B, an antimicrobial peptide derived from bovine lactoferrin. Biochemistry (1998) 37(12):4288-98.
Jones E M, Smart A, Bloomberg G, Burgess L, Millar M R. Lactoferricin, a new antimicrobial peptide. J. Applied Bacteriology 1994 77 (2) 208-14.
Kang J H, Lee M K, Kim K L, Hahm, K S. Structure-biological activity relationships of 11-residue highly basic peptide segment of bovine lactoferrin. Int. J. Peptide Prot. Res. 1996, 48, 357-363.
Komine K, Komine Y, Kuroishi T, Kobayashi J, Obara Y, Kumagai K. Small molecule lactoferrin with an inflammatory effect but no apparent antibacterial activity in mastitic mammary gland secretion. J. Vet. Med. Sci. 2005, 67 (7) 667-77.
Kuwata H, Yip T T, Yip C L, Tomita M, Hutchens, T W; Direct detection and quantitative determination of bovine lactoferricin and lactoferrin fragments in human gastric contents by affinity mass spectrometry. Advances in Exptl Med & Biology 1998, 443, 23-32.
Kuwata H, Yip T T, Tomita M, Hutchens T W. Direct evidence of the generation in human stomach of an antimicrobial peptide domain (lactoferricin) from ingested lactoferrin. Biochim Biophys Acta. (1998) 1429(1):129-41.
Kuwata H, Yamauchi K, Teraguchi S, Ushida Y, Shimokawa Y, Toida T, Hayasawa H. Functional fragments of ingested lactoferrin are resistant to proteolytic degradation in the gastrointestinal tract of adult rats. J. Nutrition 2001, 131 (8) 2121-7.
Kuwata H, Yip T T, Yamauchi K, Teraguchi S, Hayasawa H, Tomita M, Hutchens T. The survival of ingested lactoferrin in the gastrointestinal tract of adult mice. Biochemical Journal, 1998, 334 (2) 321-23.
Legrand D, Mazurier J, Metz-Boutigue M-H, Jolles J, Jo;;es P, Montreuil J & Spik G (1984). Characterization and localization of an iron-binding 18-kDa glycopeptide isolated from the N-terminal half of human lactotransferrin. Biochimica et Biophysica acta 787, 90-96.
Legrand D, Mazurier J, Aubert J P, Loucheuxlefebvre M H, Montreuil J, Spik G. Evidence for interactions between the 30 kDa N-terminal and 50 kda C-terminal tryptic fragments of human lactotransferrin. Biochemical Journal 1986, 236 (3) 839-44.
Leung, D. W., Chen, E. Y., Goeddel, D. V. A Method for Random Mutagenesis of a Defined DNA Segment Using a Modified Polymerase Chain Reaction. Technique (1989) 1:11-15.
Lindberg T, Engberg S, Jakobsson I, Lonnerdal B. Digestion of proteins in human milk, human milk fortifier, and preterm formula in infant rhesus monkeys. J. Paediatric Gastroenterology & Nutrition 1997, 24 (5) 537-43.
Longhi C, Cote M P, Bellamy W, Seganti L, Valenti P. Effect of lactoferricin B, a pepsin-generated peptide of bovine lactoferrin, on Escherichia coli HB 101 (pR1203) entry into HeLa cells. Medical Microbiology & Immunology 1994, 183 (2) 77-85.
Longhi C, Conte M P, Ranaldi S, Penta, M, Valenti P, Tinari A, Superti F, Seganti L. Apoptotic death of Listeria monocytogenes-infected human macrophages induced by lactoferricin B, a bovine lactoferrin-derived peptide. Int. J. Immunopathology & Pharmacology 2005, 18 (2) 317-25.
Lowe C, Cornish J, Callon K, Martin T J, Reid I R., Regulation of osteoblast proliferation by leukemia inhibitory factor. J Bone Miner Res. (1991) 6(12):1277-83.
Masschalck B, Van Houdt R, Michiels C W. High pressure increases bactericidal activity and spectrum of lactoferrin, lactoferricin and nisin. Int. J. Food Microbiology 2001, 64 (3) 325-32.
Masson P L, Heremans J F. Studies on lactoferrin, the iron-binding protein of secretions. Protides of the Biological Fluids 1966, 14, 115-24.
Mata L, Castillo H, Sanchez L, Puyol P, Calvo M. Effect of trypsin on bovine lactoferrin and interaction between the fragments under different conditions. J Dairy Res. (1994) 61(3):427-32.
McCann K B, Lee A, Wan J, Roginski H, Coventry M J. The effect of bovine lactoferrin and lactoferricin B on the ability of feline calicvirus (a nonvirus surrogate) and poliovirus to infect cell cultures. J. Applied Microbiology 2003, 95 (5) 1026-33.
Metz-Boutigue M H, Jolles J, Mazurier J, Schoentgen F, Legrand D, Spik G, Montreuil J, Jolles P. Human lactotransferrin: amino acid sequence and structural comparisons with other transferrins. Eur J Biochem. (1984) 145(3):659-76.
Miyauchi H, Kaino A, Shinoda I, Fukuwatari Y, Hayasawa H. Immunomodulatory effect of bovine lactoferrin pepsin hydrolysate on murine splenocytes and Peyer's patch cells. J. Dairy Science 1997, 80 (10) 2330-9.
Murdock C A & Matthews I R. Antibacterial activity of pepsin-digested lactoferrin on foodborne pathogens in buffered broth systems and ultra-high temperature milk with EDTA. J. Applied Microbiology 2002; 93 (5) 850-6.
Nguyen L T, Schibli D J, Vogel J. Structural studies and model membrane interactions of two peptides derived from bovine lactoferricin. Journal of Peptide Science 2005, 11 (7) 379-89.
Odell E W, Sarra R, Foxworthy M,. Chapple D S, Evans R W. Antibacterial activity of peptides homologous to a loop region in human lactoferrin. FEBS Lett. 1996, 382 (1/2) 175-8.
Pierce, A., Colavizza, D., Benaissa, M., Maes, P., Tartar, A., Montreuil, J. and Spik, G., Molecular cloning and sequence analysis of bovine lactotransferrin. Eur. J. Biochem. 196 (1), 177-184 (1991).
Rahman M Md, Kumara H, Shimazaki K. Growth promotional effects of bovine lactoferrin and its hydrolysate on bifidobacteria. Miruku Saiensu 2004, 53 (4) 325-327.
Rochard E, Legrand D, Mazurier J, Montreuil J, Spik G. The N-terminal domain I of human lactotransferrin binds specifically to phytohemagglutinin-stimulated peripheral blood human lymphocyte receptors. FEBS Lett. 1989, 255 (1) 201-4.
Roy M K, Kuwabara Y, Hara K, Watanabe Y, Tamai Y. Peptides from the N-terminal end of bovine lactoferrin induce apoptosis in human leukemic (HL-60) cells. J. Dairy Science 2002, 85 (9) 2065-74.
Sakai T, Kawaguchi M, sawaki K, Okubo M, Shinomiya T, Banno Y. Pepsin-digested bovine lactoferrin induces apoptotic cell death and survival mechanism in oral squamous cell carcinoma cells. Miruku Saiensu 2004, 53 (4) 254-5.
Sambrook, J.; Fritsch, E. F.; Maniatis, T. (1989). Molecular Cloning: A Laboratory Manual. Cold Spring Harbour Lab Press, Cold Spring Harbour, N.Y.
Schibli D J, Hwang P M, Vogel H J. The structure of the antomicrobial center of lactoferricin B bound to sodium dodecyl sulphate micelles. FEBS Letters 1999, 446 (2-3) 213-17.
Schmidt A M, Yan S D, Brett J, Mora R, Nowygrod R, Stem D. Regulation of human mononuclear phagocyte migration by cell surface-binding proteins for advanced glycation end products. Journal of Clinical Investigation 1993, 91 (5) 2155-68.
Schmidt A M, Mora R, Cao R,; Yan S D, Brett J, Ramakrishnan R, Tsang T C, Simionescu M, Stern D. The endothelial-cell binding-site for advanced glycation end-products consists of a complex—an integral membrane-protein and a lactoferrin-like polypeptide. Journal of Biological Chemistry, 1994, 269 (13) 9882-88.
Shimazaki K, Nagat S, Yoo Y C. Susceptibility of bovine lactoferrin to plasmin and chymosin. Agricultural and Biological Chemistry 1991, 55 (4) 1125-26.
Shimazaki K, Tanaka T, Igarashi I, Saito A, Kanbara H, Susuki N. separation of tryptic fragments of bovine lactoferrin and their ability to bind to Trypanosoma cruzi. Int. Dairy Fed. Proceedings of a seminar, Uppsala 1993, 122-130.
Shin K, Yamauchi K, Teraguchi S, Hayasawa H, Tomita M, Otsuka Y, Yamasaki S. Antibacterial activity of bovine lactoferrin and its peptides against enterohaemorrhagic Escherichia coli O157-H7. Letters in Appl. Microbiology 1998, 26 (6) 407-11.
Siciliano R, Rega B, Marchetti M, Seganti L, Antonini G, Valenti P. Bovine lactoferrin peptidic fragments involved in inhibition of herpes simplex virus type 1 infection. Biochem. Biophys. Res. Commun. 1999, 264 (1) 19-23.
Sitaram M P, McAbee D D. Isolated rat hepatocytes differentially bind and internalize bovine lactoferrin N- and C-lobes. Biochem. J. 1997, 323, 815-22.
Spik G, Brunet B, Mazurierdehaine C, Fontaine G, Montreuil J. Characterization and properties of the human and bovine lactotransferrins extracted from the feces of newborn-infants. Acta Paediatrica Scandinavica 1982, 71 (6):979-85.
Stemmer W P. DNA shuffling by random fragmentation and reassembly: in vitro recombination for molecular evolution. Proc Natl Acad Sci USA 1994; 91:10747-10751.
Strom M B, Rekdal O, Svendsen J S; Antibacterial activity of 15-residue lactoferricin derivatives. J. Peptide Research, 2000, 56 (5) 265-274.
Superti F, Siciliano R, Rega B, Giansanti F, Valenti P, Antonini G (2001) Involvement of bovine lactoferrin metal saturation, sialic acid and protein fragments in the inhibition of rotavirus infection. Biochim Biophya Acta 1528 (2-3) 107-15.
Tani, F., Iio, K., Chiba, H. and Yoshikawa, M. Isolation and characterization of opioid antagonist peptides derived from human lactoferrin. Agric. Biol. Chem. 54 (7), 1803-1810 (1990).
Tatiana A. Tatusova, Thomas L. Madden, “Blast 2 sequences—a new tool for comparing protein and nucleotide sequences”, FEMS Microbiol Lett. (1999) 174:247-250.
Tomita M, Takase, M, Wakabayashi H & Bellamy W (1994) Antimicrobial Peptides of Lactoferrin in Lactoferrin Structure and Function, pp 209-218. Eds T W Hutchens, S V Rumball, B Lonnerdal, Plenum Press, New York.
Tomita M, Wakabayashi H, Yamauchi K, Teraguchi S, Hayasawa H; Bovine lactoferrin and lactoferricin derived from milk: production and applications. Biochemistry and Cell Biology 2002, 80 (1) 109-12.
Tomita M, Bellamy W, Takase M, Yamauchi K, Wakabayashi H, Kawasi K. Potent antibacterial peptides generated by pepsin digestion of bovine lactoferrin. J. Dairy Science 1991, 74 (12) 4137-4
Tomita M, Takase M, Bellamy W, Shimamura S. A review: the active peptide of lactoferrin. Acta Paediatr. Jpn. 1994, 36, 585-91.
Troost F J, Steijns J, Saris W H, Brummer R J. Gastric digestion of bovine lactoferrin in vivo in adults. J. Nutrition 2001, 131 (8)2101-4.
Tsuji S, Hirata Y, Matsuoka K. Two apparent molecular forms of bovine lactoferrin. J Dairy Sci. (1989) 72(5):1130-6.
Turchany J M, Aley S B, Gillin F D. Giardicidal activity of lactoferrin and N-terminal peptides. Infect. Immun. 1995, 63 (11) 4550-52.
Tweedie J R, Bain H B, Day C L, Nicholson H H, Mead P E, Sheth B & Stowell K M (1994) Lactoferrin cDNA in Lactoferrin Structure and Function, pp 197-208. Eds TW Hutchens, SV Rumball, B Lonnerdal, Plenum Press, New York.
Ulvatne H, Vorland L H. Bactericidal kinetics of 3 lactoferricins against Staphylococcus aureus and Escherichia coli. Scand. J. Infectious Diseases 2001, 33 (7) 507-11.
van der Kraan M I A, Groenink J, Nazmi K, Veerman E C I, Bolscher J G M, Nieuw Amerongen A V. Lactoferrampin: a novel antimicrobial peptide in the NI-domain of bovine lactoferrin. Peptides 2004, 25 (2) 177-83.
van Veen H A, Geerts M E, van Berkel P H, Nuijens J H. The role of N-linked glycosylation in the protection of human and bovine lactoferrin against tryptic proteolysis. Eur. J. Biochem. (2004) 271(4): 678-684.
Viejo-Diaz M, Andrés M T, Pérez-Gil J, Sánchez M, Fierro J F. Potassium Efflux Induced by a New Lactoferrin-Derived Peptide Mimicking the Effect of Native Human Lactoferrin on the Bacterial Cytoplasmic Membrane. Biochemistry (Moscow) 2003, 68 (2) 217-27
Vogel H J, Schibli D J, Jing W, Lohmeier-Vogel E M, Epand R F & Epand M (2002) Towards a structure-function analysis of bovine lactoferricin and related tryptophan- and arginine-containing peptides. Biochemistry and Cell Biology 80 (1), 49-63.
Yamauchi K, Tomita M, Giehl T J, Ellison III R T. Antibacterial activity of lactoferrin and a pepsin-derived lactoferrin peptide fragment. Infect. Immun. 1993, 61 (2) 719-28.
Yoo Y C, Watanabe R, Shimazaki K I, Paik T H, Azuma I. Regulatory effect of bovine lactoferricin on the cell cycle in human monocytic THP-1 cells. In: Lactoferrin: structure, function and applications. Proceedings of the 4th International Conference on Lactoferrin, Saporo, Japan 1999, p 163-171; eds Shimazaki K, Tsuda H, Tomita M, Kuwata T, Perraudin J P; Elsevier, Amsterdam.
Yoo Y C, Watanabe, R, Koike Y, Mitobe M, Shimazki K, Watanabe S, Azuma I. Apoptosis in human leukemic cells induced by lactoferricin, a bovine milk protein-derived peptide: involvement of reactive oxygen species. Biochem. Biophys. Res Commun. 1997, 237 (3) 624-8.
Yoshida, S. and Xiuyn, Ye. Isolation of Lactoperoxidase and Lactoferrins from Bovine Milk Acid Whey by Carboxymethyl Cation Exchange Chromatography. J Dairy Sci. (1991) 74:1439-1444.
Ziere, G J, Kruijt J K, Bijsterbosch M K, van Berkel T J. Recognition of lactoferrin and aminopeptidase m-modified lactoferrin by the liver-involvement of proteoglycans and the remnant receptor. Biochem. J. 1996, 313, 289-95.

1. A method of treating or preventing a skeletal, joint or cartilage disorder comprising administering to a subject in need thereof an effective amount of at least one lactoferrin fragment or a lactoferrin hydrolysate or a mixture thereof, wherein the lactoferrin fragment is a fragment selected from, or the lactoferrin hydrolysate comprises at least one fragment selected from
(a) a truncated lactoferrin polypeptide, or
(b) an N-lobe fragment of lactoferrin, or
(c) a C-lobe fragment of lactoferrin, or
(d) a lactoferricin, or
(e) a lactoferrampin, or
(f) a fragment of SEQ ID NO.s 5, 6, 7, 8, 9, 10, 11, 12, 13, 14, 15, 16, 17, 18, 19, 20, 21, 22, 23, 24, 25, 26, 27, 28, 29, 30, 31, 32 or 33, or
(g) a functional variant of any of (a) to (f), or
(h) a functional fragment of any of (a) to (g), or
(i) a mixture of any two or more fragments selected from (a) to (h).

2. A method according to claim 1, wherein the lactoferrin is, or the lactoferrin hydrolysate comprises a truncated lactoferrin polypeptide or a functional variant or a functional fragment thereof.

3. A method according to claim 1, wherein the lactoferrin fragment is a fragment selected from, or the lactoferrin hydrolysate comprises at least one fragment selected from
(a) an N-lobe fragment of lactoferrin, or
(b) a lactoferricin, or
(c) a functional variant of (a) or (b), or
(d) a functional fragment of (a) or (b) or (c), or
(e) a mixture of any two or more fragments selected from (a) to (d).

4. A method according to claim 1, wherein the lactoferrin fragment is a fragment selected from, or the lactoferrin hydrolysate comprises at least one fragment selected from
(a) a C-lobe fragment of lactoferrin, or
(b) a lactoferrampin, or
(c) a functional variant of (a) or (b), or
(d) a functional fragment of (a) or (b) or (c), or
(e) a mixture of any two or more fragments selected from (a) to (d).

5. A method according to claim 1, wherein the lactoferrin fragment is a fragment selected from, or the lactoferrin hydrolysate comprises at least one fragment selected from
(a) a polypeptide of SEQ ID NO. 7, 8, 11, 18, 19, 21, 23, 30, 31, 32 or 33, or
(b) a functional variant of (a), or
(c) a functional fragment of (a) or (b), or
(d) a mixture of any two or more fragments selected from (a) to (c).

6. A method according to claim 1, wherein the lactoferrin fragment is a fragment selected from, or the lactoferrin hydrolysate comprises at least one fragment selected from
(a) SEQ ID NO.s 5, 6, 7, 8, 9, 10, 11, 12, 13, 14, 15, 16, 17, 18, 19, 20, 21, 22, 23, 24, 25, 26, 27, 28, 29, 30, 31, 32 or 33, or
(b) a functional variant of SEQ ID NO.s 5, 6, 7, 8, 9, 10, 11, 12, 13, 14, 15, 16, 17, 18, 19, 20, 21, 22, 23, 24, 25, 26, 27, 28, 29, 30, 31, 32 or 33, or
(c) a functional fragment SEQ ID NO.s 5, 6, 7, 8, 9, 10, 11, 12, 13, 14, 15, 16, 17, 18, 19, 20, 21, 22, 23, 24, 25, 26, 27, 28, 29, 30, 31, 32 or 33, or
(d) a mixture of any two or more fragments selected from (a) to (c).

7. A method according to claim 1, wherein the hydrolysate is a full or partial enzyme hydrolysate, a full or partial microorganism hydrolysate, a full or partial acid hydrolysate, a full or partial cyanogen bromide hydrolysate, or a mixture thereof.

8. A method according to claim 2, wherein the truncated lactoferrin polypeptide is a polypeptide selected from SEQ ID NO. 1, 2, 3 and 4, truncated by at least about 10 amino acids at the N-terminus, the C-terminus or at both the N-terminus and C-terminus of the polypeptide.

9. A method according to claim 2, wherein the truncated lactoferrin polypeptide is a polypeptide of SEQ ID NO. 20, 24 or 26, or a mixture thereof.

10. A method according to claim 3, wherein N-lobe fragment or functional fragment thereof is a polypeptide selected from SEQ ID NO. 5, 6, 9, 10, 12, 25, 27 and 29, or a mixture of any two or more thereof.

11. A method according to claim 4, wherein the C-lobe fragment or functional fragment thereof is a polypeptide selected from SEQ ID NO. 7, 8, 11, 18, 19, 21 and 23, or a mixture of any two of more thereof.

12. A method according to claim 3, wherein the lactoferricin fragment or functional fragment thereof is a polypeptide selected from SEQ ID NO. 13, 14, 15, 16, 17 and 28, or a mixture of any two or more thereof.

13. A method according to claim 4, wherein the lactoferrampin fragment is a polypeptide selected from SEQ ID NO. 30, 31, 32 and 33, or a mixture of any two or more thereof.

14. A method according to claim 7, wherein the lactoferrin hydrolysate comprises at least one fragment selected from
(a) SEQ ID NO.s 5, 6, 7, 8, 9, 10, 11, 12, 13, 14, 15, 16, 17, 18, 19, 20, 21, 22, 23, 24, 25, 26, 27, 28, 29, 30, 31, 32 or 33, or
(b) a functional variant of SEQ ID NO.s 5, 6, 7, 8, 9, 10, 11, 12, 13, 14, 15, 16, 17, 18, 19, 20, 21, 22, 23, 24, 25, 26, 27, 28, 29, 30, 31, 32 or 33, or
(c) a functional fragment SEQ ID NO.s 5, 6, 7, 8, 9, 10, 11, 12, 13, 14, 15, 16, 17, 18, 19, 20, 21, 22, 23, 24, 25, 26, 27, 28, 29, 30, 31, 32 or 33, or
(d) a mixture of any two or more fragments selected from (a) to (c).

15. A method according to claim 7, wherein the enzyme is selected from a protease, trypsin, chymotrypsin, chymosin, plasmin, pepsin, papain, a peptidase, an aminopeptidase or a mixture thereof.

16. A method according to claim 7, wherein the enzyme is trypsin and the lactoferrin is a polypeptide having the amino acid sequence of SEQ ID NO. 2; or wherein the enzyme is pepsin and the lactoferrin is a polypeptide having the amino acid sequence of SEQ ID NO. 2.

17. A method according to claim 7, wherein the hydrolysate comprises the peptides
(1) ADAVTLDGGMVF, ADAVTLDGGMVFEAGR, ADRDQYELL, ANEGLTWN, ANEGLTWNSLK, APVDAFK, CLQDGAGDVAFVK, DGKEDLIWK, DLLFKDSALGFLR, DSALGF, DSALGFLR, EKYYGYTGAFR, EPLQGAVAK, EPYFGYSGAFK, ESPQTHYY, ESPQTHYYAVAVVK, ETTVFENLPEK, FENLPEK, FGYSGAFK, FKDSALGFLR, GEADALNLDGGY, GEADALNLDGGYIY, GILRPYLSWTESLEPLQGAVAK, GSNFQLDQLQGR, GTEYVTAIANLKK, GYSGAFK, IIPMGILRPYLSWTESLEPLQGAVAK, IPSKVDSALYLGSR, KADAVTLDGGMVF, KADAVTLDGGMVF, KANEGLTWNSLK, KDSALGFLR, KGSNFQLDQLQGR, KPVTEAQSCHLAVAPNHAVVSR, LAQVPSHAVVAR, LAVAPNHAVVSR, LAVAVVK, LFGSPPGQR, LFKDSALGFLR, LGAPSITCVR, LGGRPTYEEY, LGGRPTYEEYLGTEY, LGGRPTYEEYLGTEYVTAIANLK, LGGRPTYEEYLGTEYVTAIANLKK, LGTEYVTAIANLK, LHQQALFGK, LLHQQALFGK, LRPVAAEIY, LRPVAAEIYGTK, LSWTESLEPLQGAVAK, LQGAVAK, NFQLDQLQGR, NLLFNDNTECLAK, PLQGAVAK, PQTHYYAVAVVK, PSKVDSALYLGSR, PTEGYLAVAVVK, PTYEEYLGTEYVTAIANLK, PVAAEIYGTK, PYLSWTESLEPLQGAVAK, QLDQLQGR, QVLLHQQALF, QVLLHQQALFGK, QVLLHQQALFGKNGK, SAGWIIPMGILRPY, SAGWIIPMGILRPYLSWTESLEPLQGAVAK, SFQLFGSPPGQR, SVDGKEDLIWK, SWTESLEPLQGAVAK, TESLEPLQGAVAK, TVFENLPEK, VFENLPEK, VLLHQQALFGK, VTAIANLK, WTESLEPLQGAVAK, YAVAVVK, YFGYSGAFK, YYGYTGAF and YYGYTGAFR, or a selection thereof that is able to stimulate osteoblast proliferation or inhibit osteoclast development; or
(2) AEIYGTKESPQTHY, AENRKSSKYSSL, AKLGGRPTYE, AKLGGRPTYEE, AKNLNRED, AKNLNREDF, AQEKFGKNKSRS, ARSVDGKEDL, AVVKKANEGLTWNSL, DGGMVFEAGRDPYKLRPVA, DRDQYEL, DRTAGWNIPMGL, EAGRDPYKLRPVA, EAGRDPYKLRPVAA, EAGRDPYKLRPVAAE, EIYGTKESPQTHY, EKKADAVTL, ENLPEKADRDQ, ENLPEKADRDQY, ENLPEKADRDQYE, ENLPEKADRDQYEL, ESLEPLQG, ESLEPLQGA, ESLEPLQGAV, FEAGRDPYKLRPVA, FEAGRDPYKLRPVAA, FGKNKSRS, FGSPPGQRDL, FGSPPGQRDLL, FGSPPGQRDLLF, FKCLQDGAGDVAF, FKDSALGF, FKSETKNLL, FNDNTECL, FQLFGSPPGQRDLL, FRCLAEDVGD, GSPPGQRDLL, IAEKKADAVT, IAEKKADAVTL, IPMGI, IWKLLSKAQEKFGKNKSRS, IWKLLSKAQEKFGKNKSRSFQL, IYGTKESPQTHY, KAQEKFGKNKSRS, KDSALGF, KGEADALNL, KKADAVTL, KSETKNLL, KYYGYTGA, LECIRA, LFGSPPGQRDLL, LFKDSALGF, LKNLRE, LKNLRETAE, LNLDGGY, LPEKADRDQYE, LRIPSKVD, LRIPSKVDSA, LRIPSKVDSAL, LSKAQEKFGKNKSRS, LSKAQEKFGKNKSRSFQL, LTTLKNLRE, LTTLKNLRETAE, NLDGGY, NLDGGYI, NLNREDFRL, NLPEKADRDQ, NREDFRL, PEKADRDQ, PEKADRDQYE, PEKADRDQYEL, PPGQRDLL, PYKLRPVA, QLFGSPPGQRDLL, RSDRAAHVKQVL, RSVDGKEDL, RTAGWNIPMGL, SWTESLEPLQG, TESLEPLQG, TTLKNLRETAE, VARSVDGKEDL, VFEAGRDPYKLRPVA, VFEAGRDPYKLRPVAA, VFEAGRDPYKLRPVAAE, VKETTVF, VLKGEADAL, VSRSDRAAHVKQ, VTLDGGM, VTLDGGMV, VTLDGGMVF, VVARSVDGKEDL, VVKKANEGLTW, VVKKANEGLTWNSL, VVSRSDRAAHVKQ, VVSRSDRAAHVKQVL, WAKNLNRE, WAKNLNRED, WAKNLNREDF, WIIPMGI, WNIPMGL, YGTKESPQTHY and YLGSRY, or a selection thereof that is able to stimulate osteoblast proliferation or inhibit osteoclast development.

18. A method according to claim 7, wherein the microorganism is selected from the genera Bacillus, Bifidus, Enterococcus, Lactobacillus, Lactococcus, Leuconostoc, Pediococcus, Propionbacter, Pseudomonas, Streptococcus or a mixture thereof, or wherein the acid is selected from trifluoro acetate and hydrochloric acid.

19. A method according to claim 1, wherein the disorder is osteoporosis, rheumatoid arthritis, osteoarthritis, hepatic osteodystrophy, osteomalacia, rickets, osteitis fibrosa cystica, renal osteodystrophy, osteosclerosis, osteopenia, fibrogenesis-imperfecta ossium, secondary hyperparathyrodism, hypoparathyroidism, hyperparathyroidism, chronic renal disease, sarcoidosis, glucocorticoid-induced osteoporosis, idiopathic hypercalcemia, Paget's disease, or osteogenesis imperfecta.

20. A method according to claim 19, wherein the disorder is osteoporosis, rheumatoid arthritis or osteoarthritis.

21. A method according to claim 1, wherein the lactoferrin fragment comprises a metal ion binding site that is bound to a metal ion or the lactoferrin fragment comprises two metal ion binding sites that are independently empty or bound to a metal ion.

22. A method according to claim 21, wherein the metal ion is selected from a bismuth ion, an iron ion, copper ion, chromium ion, cobalt ion, manganese ion, zinc ion, or a mixture thereof.


SEQUENCE LISTING (37 sequences)

SEQ ID NO: 1 - protein, 708 aa, Bos taurus
MKLFVPALLSLGALGLCLAAPRKNVRWCTISQPEWFKCRRWQWRMKKLGAPSITCVRRAFALECIRAIAEKKADAVTLDGGMVFEAGRDPYKLRPVAAEIYGTKESPQTHYYAVAVVKKGSNFQLDQLQGRKSCHTGLGRSAGWIIPMGILRPYLSWTESLEPLQGAVAKFFSASCVPCIDRQAYPNLCQLCKGEGENQCACSSREPYFGYSGAFKCLQDGAGDVAFVKETTVFENLPEKADRDQYELLCLNNSRAPVDAFKECHLAQVPSHAVVARSVDGKEDLIWKLLSKAQEKFGKNKSRSFQLFGSPPGQRDLLFKDSALGFLRIPSKVDSALYLGSRYLTTLKNLRETAEEVKARYTRVVWCAVGPEEQKKCQQWSQQSGQNVTCATASTTDDCIVLVLKGEADALNLDGGYIYTAGKCGLVPVLAENRKSSKHSSLDCVLRPTEGYLAVAVVKKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLNREDFRLLCLDGTRKPVTEAQSCHLAVAPNHAVVSRSDRAAHVKQVLLHQQALFGKNGKNCPDKFCLFKSETKNLLFNDNTECLAKLGGRPTYEEYLGTEYVTAIANLKKCSTSPLLEACAFLTR

SEQ ID NO: 2 - protein, 689 aa, Bos taurus
APRKNVRWCTISQPEWFKCRRWQWRMKKLGAPSITCVRRAFALECIRAIAEKKADAVTLDGGMVFEAGRDPYKLRPVAAEIYGTKESPQTHYYAVAVVKKGSNFQLDQLQGRKSCHTGLGRSAGWIIPMGILRPYLSWTESLEPLQGAVAKFFSASCVPCIDRQAYPNLCQLCKGEGENQCACSSREPYFGYSGAFKCLQDGAGDVAFVKETTVFENLPEKADRDQYELLCLNNSRAPVDAFKECHLAQVPSHAVVARSVDGKEDLIWKLLSKAQEKFGKNKSRSFQLFGSPPGQRDLLFKDSALGFLRIPSKVDSALYLGSRYLTTLKNLRETAEEVKARYTRVVWCAVGPEEQKKCQQWSQQSGQNVTCATASTTDDCIVLVLKGEADALNLDGGYIYTAGKCGLVPVLAENRKSSKHSSLDCVLRPTEGYLAVAVVKKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLNREDFRLLCLDGTRKPVTEAQSCHLAVAPNHAVVSRSDRAAHVKQVLLHQQALFGKNGKNCPDKFCLFKSETKNLLFNDNTECLAKLGGRPTYEEYLGTEYVTAIANLKKCSTSPLLEACAFLTR

SEQ ID NO: 3 - protein, 711 aa, Homo sapiens [sequence omitted]

SEQ ID NO: 4 - protein, 692 aa, Homo sapiens
GRRRRSVQWCAVSQPEATKCFQWQRNMRKVRGPPVSCIKRDSPIQCIQAIAENRADAVTLDGGFIYEAGLAPYKLRPVAAEVYGTERQPRTHYYAVAVVKKGGSFQLNELQGLKSCHTGLRRTAGWNVPIGTLRPFLNWTGPPEPIEAAVARFFSASCVPGADKGQFPNLCRLCAGTGENKCAFSSQEPYFSYSGAFKCLRDGAGDVAFIRESTVFEDLSDEAERDEYELLCPDNTRKPVDKFKDCHLARVPSHAVVARSVNGKEDAIWNLLRQAQEKFGKDKSPKFQLFGSPSGQKDLLFKDSAIGFSRVPPRIDSGLYLGSGYFTAIQNLRKSEEEVAARRARVVWCAVGEQELRKCNQWSGLSEGSVTCSSASTTEDCIALVLKGEADAMSLDGGYVYTAGKCGLVPVLAENYKSQQSSDPDPNCVDRPVEGYLAVAVVRRSDTSLTWNSVKGKKSCHTAVDRTAGWNIPMGLLFNQTGSCKFDEYFSQSCAPGSDPRSNLCALCIGDEQGENKCVPNSNERYYGYTGAFRCLAENAGDVAFVKDVTVLQNTDGNNNEAWAKDLKLADFALLCLDGKRKPVTEARSCHLAMAPNHAVVSRMDKVERLKQVLLHQQAKFGRNGSDCPDKFCLFQSETKNLLFNDNTECLARLHGKTTYEKYLGPQYVAGITNLKKCSTSPLLEACEFLRK

SEQ ID NO: 5 - protein, 333 aa, Artificial
  Recombinant human lactoferrin N-lobe
GRRRRSVQWCAVSQPEATKCFQWQRNMRKVRGPPVSCIKRDSPIQCIQAIAENRADAVTLDGGFIYEAGLAPYKLRPVAAEVYGTERQPRTHYYAVAVVKKGGSFQLNELQGLKSCHTGLRRTAGWNVPIGTLRPFLNWTGPPEPIEAAVARFFSASCVPGADKGQFPNLCRLCAGTGENKCAFSSQEPYFSYSGAFKCLRDGAGDVAFIRESTVFEDLSDEAERDEYELLCPDNTRKPVDKFKDCHLARVPSHAVVARSVNGKEDAIWNLLRQAQEKFGKDKSPKFQLFGSPSGQKDLLFKDSAIGFSRVPPRIDSGLYLGSGYFTAIQNLR

SEQ ID NO: 6 - protein, 280 aa, Bos taurus
APRKNVRWCTISQPEWFKCRRWQWRMKKLGAPSITCVRRAFALECIRAIAEKKADAVTLDGGMVFEAGRDPYKLRPVAAEIYGTKESPQTHYYAVAVVKKGSNFQLDQLQGRKSCHTGLGRSAGWIIPMGILRPYLSWTESLEPLQGAVAKFFSASCVPCIDRQAYPNLCQLCKGEGENQCACSSREPYFGYSGAFKCLQDGAGDVAFVKETTVFENLPEKADRDQYELLCLNNSRAPVDAFKECHLAQVPSHAVVARSVDGKEDLIWKLLSKAQEKFGK

SEQ ID NO: 7 - protein, 345 aa, Bos taurus
VVWCAVGPEEQKKCQQWSQQSGQNVTCATASTTDDCIVLVLKGEADALNLDGGYIYTAGKCGLVPVLAENRKSSKHSSLDCVLRPTEGYLAVAVVKKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLNREDFRLLCLDGTRKPVTEAQSCHLAVAPNHAVVSRSDRAAHVKQVLLHQQALFGKNGKNCPDKFCLFKSETKNLLFNDNTECLAKLGGRPTYEEYLGTEYVTAIANLKKCSTSPLLEACAFLTR

SEQ ID NO: 8 - protein, 405 aa, Bos taurus
SFQLFGSPPGQRDLLFKDSALGFLRIPSKVDSALYLGSRYLTTLKNLRETAEEVKARYTRVVWCAVGPEEQKKCQQWSQQSGQNVTCATASTTDDCIVLVLKGEADALNLDGGYIYTAGKCGLVPVLAENRKSSKHSSLDCVLRPTEGYLAVAVVKKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLNREDFRLLCLDGTRKPVTEAQSCHLAVAPNHAVVSRSDRAAHVKQVLLHQQALFGKNGKNCPDKFCLFKSETKNLLFNDNTECLAKLGGRPTYEEYLGTEYVTAIANLKKCSTSPLLEACAFLTR

SEQ ID NO: 9 - protein, 282 aa, Bos taurus
APRKNVRWCTISQPEWFKCRRWQWRMKKLGAPSITCVRRAFALECIRAIAEKKADAVTLDGGMVFEAGRDPYKLRPVAAEIYGTKESPQTHYYAVAVVKKGSNFQLDQLQGRKSCHTGLGRSAGWIIPMGILRPYLSWTESLEPLQGAVAKFFSASCVPCIDRQAYPNLCQLCKGEGENQCACSSREPYFGYSGAFKCLQDGAGDVAFVKETTVFENLPEKADRDQYELLCLNNSRAPVDAFKECHLAQVPSHAVVARSVDGKEDLIWKLLSKAQEKFGKNK

SEQ ID NO: 10 - protein, 284 aa, Bos taurus
APRKNVRWCTISQPEWFKCRRWQWRMKKLGAPSITCVRRAFALECIRAIAEKKADAVTLDGGMVFEAGRDPYKLRPVAAEIYGTKESPQTHYYAVAVVKKGSNFQLDQLQGRKSCHTGLGRSAGWIIPMGILRPYLSWTESLEPLQGAVAKFFSASCVPCIDRQAYPNLCQLCKGEGENQCACSSREPYFGYSGAFKCLQDGAGDVAFVKETTVFENLPEKADRDQYELLCLNNSRAPVDAFKECHLAQVPSHAVVARSVDGKEDLIWKLLSKAQEKFGKNKSR

SEQ ID NO: 11 - protein, 407 aa, Bos taurus
SRSFQLFGSPPGQRDLLFKDSALGFLRIPSKVDSALYLGSRYLTTLKNLRETAEEVKARYTRVVWCAVGPEEQKKCQQWSQQSGQNVTCATASTTDDCIVLVLKGEADALNLDGGYIYTAGKCGLVPVLAENRKSSKHSSLDCVLRPTEGYLAVAVVKKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLNREDFRLLCLDGTRKPVTEAQSCHLAVAPNHAVVSRSDRAAHVKQVLLHQQALFGKNGKNCPDKFCLFKSETKNLLFNDNTECLAKLGGRPTYEEYLGTEYVTAIANLKKCSTSPLLEACAFLTR

SEQ ID NO: 12 - protein, 15 aa, Artificial
  Synthetic human lactoferrin N-lobe peptide
CPDNTRKPVDKFKDC

SEQ ID NO: 13 - protein, 25 aa, Bos taurus
FKCRRWQWRMKKLGAPSITCVRRAF

SEQ ID NO: 14 - protein, 26 aa, Bos taurus
FKCRRWQWRMKKLGAPSITCVRRAFA

SEQ ID NO: 15 - protein, 47 aa, Homo sapiens
GRRRRSVQWCAVSQPEATKCFQWQRNMRKVRGPPVSCIKRDSPIQCI

SEQ ID NO: 16 - protein, 11 aa, Artificial
  Synthetic bovine lactoferricin peptide
RRWQWRMKKLG

SEQ ID NO: 17 - protein, 15 aa, Artificial
  Synthetic bovine lactoferricin peptide
FKCRRWQWRMKKLGA

SEQ ID NO: 18 - protein, 348 aa, Bos taurus
YTRVVWCAVGPEEQKKCQQWSQQSGQNVTCATASTTDDCIVLVLKGEADALNLDGGYIYTAGKCGLVPVLAENRKSSKHSSLDCVLRPTEGYLAVAVVKKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLKREDFRLLCLDGTRKPVTEAQSCHLAVAPNHAVVSRSDRAAHVEQVLLHQQALFGKNGKNCPDKFCLFKSETKNLLFNDNTECLAKLGGRPTYEEYLGTEYVTAIANLKKCSTSPLLEACAFLTR

SEQ ID NO: 19 - protein, 335 aa, Bos taurus
YTRVVWCAVGPEEQKKCQQWSQQSGQNVTCATASTTDDCIVLVLKGEADALNLDGGYIYTAGKCGLVPVLAENRKSSKHSSLDCVLRPTEGYLAVAVVKKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLKREDFRLLCLDGTRKPVTEAQSCHLAVAPNHAVVSRSDRAAHVEQVLLHQQALFGKNGKNCPDKFCLFKSETKNLLFNDNTECLAKLGGRPTYEEYLGTEYVTAIANLKKCS

SEQ ID NO: 20 - protein, 681 aa, Bos taurus
CTISQPEWFKCRRWQWRMKKLGAPSITCVRRAFALECIRAIAEKKADAVTLDGGMVFEACRDPYKLRPVAAEIYGTKESPQTHYYAVAVVKKGSNFQLDQLQGRKSCHTGLGRSAGWIIPMGILRPYLSWTESLEPLQGAVAKFFSASCVPCIDRQAYPNLCQLCKGEGENQCACSSREPYFGYSGAFKCLQDGAGDVAFVKETTVFENLPEKADRDQYELLCLNNSRAPVDAFKECHLAQVPSHAVVARSVDGKEDLIWKLLSKAQEKFGKNKSRSFQLFGSPPGQRDLLFKDSALGFLRIPSKVDSALYLGSRYLTTLKNLRETAEEVKARYTRVVWCAVGPEEQKKCQQWSQQSGQNVTCATASTTDDCIVLVLKGEADALNLDGGYIYTAGKCGLVPVLAENRKSSKHSSLDCVLRPTEGYLAVAVVKKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLNREDFRLLCLDGTRKPVTEAQSCHLAVAPNHAVVSRSDRAAHVKQVLLHQQALFGKNGKNCPDKFCLFKSETKNLLFNDNTECLAKLGGRPTYEEYLGTEYVTAIANLKKCSTSPLLEACAFLTR

SEQ ID NO: 21 - protein, 18 aa, Bos taurus
  misc_feature (7)..(7): Xaa can be any naturally occuring amino acid
YTRVVWXAVGPEEQKKXQ

SEQ ID NO: 22 - protein, 14 aa, Bos taurus
MKLFVPALLSLGAL

SEQ ID NO: 23 - protein, 61 aa, Bos taurus
ESTADWAKNLNREDFRLLCLDGTRKPVTEAQSCHLAVAPNHAVVSRSDRAAHVKQVLLHQQ

SEQ ID NO: 24 - protein, 112 aa, Bos taurus
EKFGKNKSRSFQLFGSPPGQRDLLFKDSALGFLRIPSKVDSALYLGSRYLTTLKNLRESAAEEVKARYTRVVWCAVGPEEQKKCQQWSQQSGQNVTCATASTTDDCIVLVLK

SEQ ID NO: 25 - protein, 110 aa, Bos taurus
SPQTHYYAVAVVKKGSNFQLDQLQGRKSCHTGLGRSAGWIIPMGILRPYLSWTESLEPLQGAVAKFFSASCVPCIDRQAYPNLCQLCKGEGENQCACSSREPYFGYSGAF

SEQ ID NO: 26 - protein, 117 aa, Bos taurus
NLPEKADRDQYELLCLNNSRAPVDAFKECHLAQVPSHAVVARSVDGKEDLIWKLLSKAQEKFGKNKSRSFQLFGSPPGQRDLLFKDSALGFLRIPSKVDSALYLGSRYLTTLKNLRE

SEQ ID NO: 27 - protein, 164 aa, Bos taurus
KKADAVTLDGGMVFEAGRDPYKLRPVAAEIYGTKESPQTHYYAVAVVKKGSNFQLDQLQGRKSCHTGLGRSAGWVIPMGILRPYLSWTESLEPLQGAVAKFFSASCVPCIDRQAYPNLCQLCKGEGENQCACSSREPYFGYSGAFKCLQDGAGDVAFVKETTVF

SEQ ID NO: 28 - protein, 26 aa, Bos taurus
MFKCRRWQWRMKKLGAPSITCVRRAF

SEQ ID NO: 29 - protein, 33 aa, Ovis aries
APRKNVRWCAISPPEGSKCYQWQKKMRKLGRPL

SEQ ID NO: 30 - protein, 17 aa, Bos taurus
WKLLSKAQEKFGKNKSR

SEQ ID NO: 31 - protein, 11 aa, Bos taurus
WKLLSKAQEKF

SEQ ID NO: 32 - protein, 6 aa, Bos taurus
GKNKSR

SEQ ID NO: 33 - protein, 16 aa, Bos taurus
WKLLSKAQEKFGKNKS

SEQ ID NO: 34 - protein, 31 aa, Artificial Sequence
  Synthetically generated Human kaliocin-1 by Fmoc chemistry
FFSASCVPGADKGQFPNLCRLCAGTGENKCA

SEQ ID NO: 35 - protein, 23 aa, Artificial Sequence
  Synthetically generated Lactoferricin derived peptide by Fmoc chemistry
TKCFQWQRNMRKVRGPPVSCIKR

SEQ ID NO: 36 - protein, 11 aa, Artificial Sequence
  Synthetically generated Bovine lactoferricin peptide by Fmoc chemistry
RRWQWRMKKLG

SEQ ID NO: 37 - protein, 17 aa, Artificial Sequence
  Synthetically generated Lactoferrampin by Fmoc chemistry
WKLLSKAQEKFGKNKSR